(19) United States
(12) Patent Application Publication
(10) Pub. No.: US 2026/0295258 A1
(43) Pub. Date: Oct. 1, 2026

(54) CLOSED-LOOP EXTERNALLY POWERED ELECTRICAL CORTICAL STIMULATIONS (XCS)

(71) Applicant: Motif Neurotech, Inc., Houston, TX (US)

(72) Inventors: Brandon Thio, Houston, TX (US); Ameya Nanivadekar, Houston, TX (US); Landan Mintch, Houston, TX (US); Amanda Singer, Houston, TX (US); Elizabeth Commissaris, Houston, TX (US); Jacob Robinson, Houston, TX (US); Steven Goetz, Houston, TX (US)

(73) Assignee: Motif Neurotech, Inc., Houston, TX (US)

(21) Appl. No.: 19/559,866

(22) Filed: Mar. 6, 2026

Related U.S. Application Data
(60) Provisional application No. 63/777,561, filed on Mar. 25, 2025.

(51) Int. Cl. A61N 1/36; A61B 5/00; A61N 1/05; G16B 5/20; G16H 20/30

(52) U.S. Cl. CPC A61N 1/3606; A61B 5/4836; A61N 1/0531; G16B 5/20; G16H 20/30

(57) ABSTRACT

The present disclosure relates dynamic closed-loop neuromodulation techniques to induce an externally powered electrical cortical stimulation (XCS) configured for various applications by leveraging real-time neural data of a subject. The neural data may be collected by one or more neuromodulation devices, placed epidurally through surgical implantation into a skull. A brain state may be estimated by processing the neural data via analytical techniques. Based on the predicted brain state, a set of stimulation parameters, associated with the XCS for a cortical region, may be determined. This determination may rely on a state transition model, configured to learn how brain transitions from one brain state to another based on the ongoing neural activity. The brain responses to applied XCS may be monitored from the neural data collected in real-time. Based on the monitoring, one or more stimulation parameters may be dynamically adjusted for each XCS in a closed loop.

### CROSS-REFERENCE TO RELATED APPLICATIONS

This application claims the benefit of U.S. Provisional Patent Application No. 63/777,561 filed on Mar. 25, 2025. The entire disclosures of the aforementioned applications are incorporated by reference herein in their entireties for all purposes.

### BACKGROUND

Neuromodulation is an evolving field of medical science that involves the application of targeted electrical, chemical, or magnetic stimuli to the nervous system to regulate neural or nerve activities. These neuromodulation techniques induce stimulation within specific regions of the brain, spinal cord, or peripheral nerves by using devices that deliver controlled electrical, chemical, or magnetic signals. The parameters of this stimulation—such as intensity, frequency, pulse width, and duration—contribute to determining the therapeutic effects. Empirical research suggests that neuromodulation therapies show promising outcomes in treating a range of conditions, including chronic pain, neurological disorders, and psychiatric diseases, particularly when conventional therapies such as medications or surgery may be less effective or cause significant undesirable side effects. Neuromodulation therapies are typically categorized into invasive and non-invasive methods. Non-invasive methods rely on external devices, while invasive methods often require surgical implantation of devices that directly deliver stimulation to the nervous system.

Neuromodulation therapies rely heavily on specific stimulation parameters (e.g., intensity, frequency, pulse width, and duration) to achieve targeted therapeutic outcomes. A stimulation delivered to the nervous system is delivered based on these parameters, in accordance with the individual needs of each subject. However, challenges may arise due to the dynamic nature of neural responses, which can fluctuate over time as a result of changes in the subject's physiological condition, disease progression, or external factors such as environmental conditions. In particular, invasive neuromodulation devices, which are surgically implanted, may be affected by such factors, altering performance of neuromodulation devices and requiring adjustments to the stimulation parameters. Inadequate or excessive stimulation may lead to suboptimal therapeutic outcomes, side effects (e.g., twitching, muscle spasm, discomfort) or even adverse effects, further complicating the treatment process. Moreover, the stimulation requirements for each subject may vary significantly, highlighting personalized dosing and continuous monitoring. Therefore, real-time assessment of neural responses and constant adjustments to stimulation parameters may help in maintaining effective therapy and tailoring to the evolving condition of individuals.

### SUMMARY

Certain aspects of the present disclosure relate to dynamic closed-loop neuromodulation techniques to trigger externally powered electrical cortical stimulation (XCS) configured to perform a predefined treatment therapy based on neural data of a subject collected in real-time. The predefined treatment therapy may include e.g., enhancing target engagement, maintaining target engagement, reducing side effects of the treatment therapy (e.g., twitching, drowsiness) and/or preventing a relapse. Target engagement may refer to a process by which a stimulation (e.g., electrical, magnetic, or chemical stimulus) interacts with neural activities in the brain, where the stimulation modulates neuronal firing patterns, connectivity, or signaling pathways. The closed-loop indicates that the process is dynamic and adaptive, where the triggered XCS is adjusted by fine-tuning one or more stimulation parameters based on real-time feedback from the neural data including responses to the stimulation.

The neural data, capturing brain activities of the subject, may be collected in real-time continuously (or at specified time intervals during the triggered stimulation) from one or more neuromodulation devices that are surgically implanted into a skull of the subject via a burr hole procedure. The one or more neuromodulation devices may be placed epidurally (e.g., resting above dura mater without direct contact with the brain) over one or more cortical regions to deliver electrical stimulations (interchangeably used herein with XCS). Following the implantation, the skin and the underlying tissues may then be repositioned and sutured, enclosing the one or more neuromodulation devices within the epidural space. In some aspects, the one or more neuromodulation devices configured to sense (or record neural activities and responses) and inducing stimulation (or XCS) are wirelessly powered by one or more magnetic coils that are configured externally within a headset. Each of at least one of the one or more magnetic coils may be, may include or may be replaced or supplemented with a magnetic transceiver.

The neural data, representing continuous time-series data, may include one or more of: local field potentials (LFPs), which refer to electrical activities within a local proximity of a neuromodulation device; stimuli-evoked potentials (SEPs), which are brain responses to stimuli e.g., (XCS or an external stimulus); and event-related potentials (ERPs), which are brain responses linked to specific events such as sensory stimuli (e.g., tactile, visual or auditory stimuli) or cognitive processes (e.g., engagement or alertness, memory tasks), all collected from the one or more neuromodulation devices before, during, and/or after therapeutic XCS delivery.

The collected neural data may be processed to estimate a brain state to evaluate whether the brain is in a state that would be conducive to responding well to XCS. The brain state may be estimated by processing the neural data using one or more analytical techniques e.g. recruitment mapping curves for SEPs, principal component decomposition (PCD) for LFPs, time domain measures (e.g., duration of neural activity burst or changes in neural signature over time), or spectral analysis such as determination of a power spectral density. These analytical techniques may be configured to either identify patterns or quantify brain activity associated with different brain states. For example, steepness and a shape of a recruitment curve (e.g., obtained by plotting recorded responses or the neural data against the stimulation levels) may indicate the brain state that may be classified e.g., as a responsive state (indicating active engagement or alertness), a non-responsive state (indicating reduced engagement or inactivity), a healthy state, an unhealthy state (e.g., relapsed state) or other functional modes. Similarly, a presence (or an absence) of power spectral components or the dynamic processing of power spectral features may similarly indicate various brain states of the subjects. These brain states may reflect likely receptivity to therapeutic interventions, or via excitability measures, the probability of an adverse event.

Based on the predicted brain state, a set of stimulation parameters, associated with each stimulation of one or more stimulations that are configured for the one or more cortical regions, may be determined. This determination may rely on a state transition model that is configured to learn how the brain transitions from one brain state to another over time based on the ongoing neural activity (or the neural data). The state transition model may learn from past brain states (or historical data) to understand how different stimulation parameters (e.g., intensity, pulse width, frequency, number of pulses) affect brain state transitions and corresponding stimulation outcomes. Subsequently, the one or more stimulations may be triggered over the one or more cortical regions of the subject via the one or more neuromodulation devices. Each stimulation may be generated with respect to the determined set of stimulation parameters, configured to perturb (or modulate) the brain activities with respect to the predefined treatment therapy (or an XCS therapy). The state transition model may be incorporated directly within the neuromodulation devices, or alternatively, within a controller integrated into the headset. In some aspects, the state transition model may comprise a Hidden Markov Model (HMM).

In some aspects of the present disclosure, one or more neural responses to the applied one or more stimulations (or XCSs) may be monitored from the neural data collected in real-time via the one or more neuromodulation devices. Based on the neural responses, the one or more stimulation parameters may be dynamically adjusted for each stimulation of the one or more stimulations via the controller. These adjustments may be configured to perform the predefined treatment therapy by guiding the brain activities through targeted brain state transitions using the insights gained from the learned state transition model. The triggered one or more stimulations may comprise a single pulse or a sequence of pulses (e.g., a burst or a train) with a predefined amplitude (or intensity), number of pulses, time interval between pulses, and pulsewidth depending upon the specific treatment therapy e.g., enhancing target engagement or reducing side effects of the treatment therapy. In some aspects, for dynamically adjusting the stimulation parameters in a closed loop, the neural data may be recorded at specified intervals (e.g., during interburst intervals of a triggered XCS).

In some examples, neural responses may be recorded during time intervals between each pulse or during interburst intervals between burst of pulses associated with the one or more triggered stimulations. Upon determining that the recorded neural response reached a predefined threshold that indicates a targeted neural activation, the triggered stimulation may be dynamically terminated thereby skipping the remaining pulses. This approach may help avoiding potential adverse effects associated with excessive or prolonged stimulation and reducing power consumption by preventing unnecessary pulses, thus extending the operational life of implantable devices.

Additionally, in some aspects, the state transition model may integrate quantifiable features e.g., baseline features and evoked features that are derived from biophysical data. Raw biophysical data including, but not limited to, electrocardiography (ECG), electrodermal activity (EDA), electrooculography (EOG), electromyography (EMG) may be acquired non-invasively from the subject prior to, or during the XCS therapy. The baseline features calculated from the biophysical data in the absence of a stimulus may include e.g., EEG peak alpha frequency, EEG theta power, EEG broadband local field potential (LFP) and/or ECG baseline heart rate and heart rate variability. The evoked features calculated or derived from the biophysical data in response to a stimulus (e.g., XCS or an external stimulus such as transcranial magnetic stimulation (TMS) or transcranial electrical stimulation (TES)) may include e.g. EEG peak evoked potential amplitude and EEG peak evoked potential timing. This integration may enhance the ability of the state transition model to map neurocardiac coupling and brain state from extracranial recordings, both of which may be incorporated into the state transition model to improve target engagement and the precision of stimulation parameters.

In some aspects, the disclosed techniques may include predicting a subject candidacy for the externally powered electrical cortical stimulation (XCS). The subject candidacy may correspond to a likelihood of the subject to benefit from XCS therapy, prior to the implantation of the one or more neuromodulation devices. The prediction techniques may include collecting non-invasive biophysical signals (or biophysical data) in the absence of external stimuli (or stimulation), referred to as a first neural data. Similarly, the techniques may involve collecting the biophysical data in the presence of external stimulation, such as TES or TMS, resulting in a second neural data. From the first neural data, baseline features may be estimated, while evoked features may be derived from the second neural data. These features may be correlated with therapeutic outcomes following XCS therapy, providing an insight into how the brain responds to stimulation. Additionally, these features may assist in assessing the connectivity state of the brain, which reflects the degree of synchronization and communication between cortical regions. Using both baseline and evoked features, a classification model may predict the subject candidacy by analyzing patterns across various signal types and/or extracted features, such as EEG metrics, heart rate variability, and TMS responses, and comparing them to known therapeutic outcomes from prior subjects who have received TMS or XCS treatments.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present disclosure is described in conjunction with the appended figures:

FIG. 1 illustrates an exemplary procedure of implanting a neuromodulation device, in accordance with some aspects of the present disclosure.

FIG. 2 depicts an illustration of a neuromodulation system comprising one or more neuromodulation devices implanted over one or more cortical regions of a subject, in accordance with some aspects of the present disclosure.

FIG. 3 illustrates a block diagram for predicting subject candidacy for an externally powered electrical cortical stimulation (XCS) therapy, in accordance with some aspects of the present disclosure.

FIG. 4A depicts an exemplary illustration for providing closed-loop or “human-in-the-loop” XCS stimulations aimed at enhancing target engagement for the XCS therapy, in accordance with some aspects of the present disclosure.

FIG. 4B illustrates an exemplary curve demonstrating a closed-loop neuromodulation technique that is configured to dynamically adjust stimulation parameters based on neural data (or neural recordings), in accordance with some aspects of the present disclosure.

FIG. 4C illustrates an exemplary graph depicting a relationship between activation thresholds to the number of pulses required to elicit a targeted neural response across multiple experimental sessions, in accordance with some aspects of the present disclosure.

FIG. 4D shows illustrative examples of stimulation bursts, each comprising sequence of pulses, in accordance with some aspects of the present disclosure.

FIG. 5 illustrates exemplary graphs for the closed-loop neuromodulation technique that is configured to maintain target engagement by dynamically adjusting the stimulation parameters.

FIG. 6 illustrates exemplary graphs for reducing side effects of the disclosed neuromodulation techniques, in accordance with some aspects of the present disclosure.

FIG. 7 illustrates an exemplary block diagram for predicting a relapse risk for the subject undergoing the XCS therapy, in accordance with some aspects of the present disclosure.

FIG. 8 illustrates an exemplary graph of time-series progression of brain states derived from periodically sampled neural data and non-neural data to dynamically adjust therapeutic modes in response to the changes in brain states.

FIG. 9 illustrates an exemplary workflow that relates to the closed-loop neuromodulation techniques to induce the externally powered electrical cortical stimulation (XCS).

### DETAILED DESCRIPTION

Some embodiments of the present disclosure relate to techniques for delivering human-in-the-loop, externally powered electrical cortical stimulation (XCS) for various treatment therapies by leveraging real-time neural data collected from the subject. Various treatment therapies (interchangeably used herein with XCS therapies) may include enhancing or maintaining target engagement, reducing side effects of a treatment therapy, and reducing a relapse risk. In an XCS therapy, one or more stimulation parameters of a triggered XCS may be dynamically adjusted based on feedback from the real-time neural data. This approach, including closed-loop XCS therapy, may be used in a range of therapeutic areas, such as neurorehabilitation, cognitive enhancement, mood regulation, and the treatment of neurological disorders such as epilepsy, Parkinson's disease, and depression. The term “human-in-the-loop” indicates that the process is dynamic and adaptive, in which one or more stimulation parameters of a triggered XCS (or stimulation) are continuously adjusted based on real-time feedback from the neural data capturing brain response to the stimulation. The neural data may be collected (or recorded) via a set of neuromodulation devices, or alternatively, one or more strategically selected neuromodulation devices that are implanted surgically into a skull of the subject by drilling a burr hole, thereby creating a brain mesh or a neural mesh of nodes.

Following the implantation, the skin and the underlying tissues may then be repositioned and sutured, enclosing the set of neuromodulation devices within cranium and above the epidural space. In some aspects, a digitally programmable over brain therapeutics (DOT) is employed as the neuromodulation device to collect (or record) neural data from a cortical region of the brain. These devices may be implanted within the skull epidurally (e.g., resting above dura mater without direct contact with the brain) over the cortical regions. The DOT may include stimulation electrodes, configured to deliver a precise electrical impulse or an electrical stimulation (interchangeably used herein with XCS) to the targeted cortical region without penetrating the dura mater. The DOT may be dynamically programmable, via a controller that may be configured externally in a headset, enabling dynamic adjustment of the one or more stimulation parameters based on the brain responses in real-time. The stimulation parameters of XCS refer to the specific settings that control the stimulation, including intensity, frequency, pulsewidth, waveform, duty cycle, and polarity. These stimulation parameters may be adjusted either separately or in combination to improve the therapeutic effects.

In some aspects, the brain mesh of implanted neuromodulation devices (or DOTs) may form a distributed neuromodulation system capable of interacting in real-time, where each DOT may perform a variety of functions, including stimulating the brain by applying XCS, recording the neural data and neural responses, communicating with neighboring DOTs (e.g., through radio frequency protocols), and executing local computations to fine-tune the one or more stimulation parameters based on real-time data. The distributed neuromodulation system may further include a magnetic coil system, embedded within the headset, comprising a single magnetic coil or an array of magnetic coils configured to wirelessly power and/or program the neuromodulation devices. In addition to direct communication between the DOTs, indirect communication may be employed via the headset, which acts as a central hub for powering and controlling the entire neuromodulation system comprising magnetic coils and neuromodulation devices.

The neural data, representing continuous time-series data, may include one or more of: local field potentials (LFPs), which refer to electrical activities within a local proximity of the DOT; stimuli-evoked potentials (SEPs), which are brain responses to a stimulus e.g., (XCS or an external stimulus); and event-related potentials (ERPs), which are brain responses linked to specific events such as sensory stimuli (e.g., tactile, visual or auditory stimuli) or cognitive processes (e.g., engagement or alertness, memory tasks) collected via DOTs before, during, and/or after therapeutic XCS delivery.

In some aspects, the neural data may be processed to derive brain state estimates or neural signatures, which correspond to distinct brain states, including e.g., a healthy brain state, an unhealthy or relapsed brain state, and a relapse risk brain state. These brain states reflect brain health of the subject and the potential for therapeutic interventions to be effective or durable. To quantify neural signatures of different brain states, metrics such as brain wave frequencies (e.g., alpha, beta, theta), functional connectivity, and event-related potentials (ERPs) may be extracted from the neural data. Predefined thresholds, established from normative and clinical data, may help classify these states. For example, a healthy brain state may be characterized by balanced alpha (8-12 Hz) and beta (13-30 Hz) wave activity, indicating effective cognitive and emotional functioning. A relapse risk state may show subtle changes, e.g., elevated theta waves (4-8 Hz) or reduced connectivity in self-regulation regions, signaling vulnerability. In contrast, an unhealthy or relapse state may involve more significant disruptions, such as lower alpha power and impaired brain network connectivity, particularly in areas linked to decision-making and emotional control. These thresholds may assist in tracking shifts in neural data to identify different brain states, helping to detect increasing risk levels and facilitating early intervention before relapsing or further decline takes place.

The derived brain states may be computed by leveraging the neural data from either a single DOT or from a strategically selected subset of DOTs in the brain mesh, depending on the specific application. This cross-DOT communication can be leveraged to deliver phase-locked stimulation, where the stimulation of one DOT is timed to lead or follow the stimulation of another DOT, or more commonly, the neural responses induced by another DOT, according to a specified phase relationship. For instance, a first DOT might induce a neural response in the beta frequency range (13-30 Hz), while a second DOT may entrain (e.g., align timing and/or phase) a higher-frequency stimulus (such as gamma at 50-70 Hz) to follow the first response. This sequence of stimulation mirrors patterns seen in healthy neural systems, where such frequency-specific interactions between brain regions contribute to encoding and transferring information.

In some examples, neural data is processed using various analytical techniques to identify patterns or quantify brain activities that correlate with different brain states. Techniques such as recruitment curve mapping for stimuli-evoked potentials (SEPs) and principal component decomposition for local field potentials (LFPs) can simplify complex neural recordings into more interpretable forms. Other techniques may include time-domain measures, such as the duration of bursting neural activity or changes in neural signatures over time, as well as spectral analysis, such as determining power spectral density. Recruitment curve mapping, for example, plotted from time-series data (e.g., SEPs), estimates brain states by analyzing how different brain regions respond to different patterns of a stimulation burst (e.g., including number of pulses of stimulation delivered, the number of bursts of stimulation delivered, the timing of the pulses or the bursts or varying levels of stimulation. This technique helps identify activation thresholds, providing insight into brain engagement, alertness, and other cognitive or therapeutic states.

Principal component decomposition, on the other hand, reduces the complexity of neural data by identifying the most significant components that explain the variance in neural activity, thus isolating the primary neural dynamics relevant to brain state estimation. For some techniques, a lower activation threshold with stronger responses suggests greater engagement, while a higher threshold with weaker responses may indicate reduced engagement. Similarly, the presence or absence of power spectral components, or the dynamic processing of power spectral features, may signal different brain states.

Based on the predicted brain state, a set of stimulation parameters for each stimulation may be determined, which may be applied to actively perturb brain activity for the targeted cortical regions and promote transitions between different states. This process may be guided by a state transition model, such as a Hidden Markov Model (HMM), which learns how the brain shifts from one state to another over time based on ongoing neural activity. The model may utilize data from past data e.g., previous brain states and stimulation outcomes to determine how various stimulation parameters (e.g., intensity, pulse width, number of pulses, frequency) influence brain state transitions. The state transitional model may track dynamic changes between brain states with and without stimulation, enabling adaptive learning. Over time, the stimulation patterns may be fine-tuned to guide the brain through targeted transitions. The state transition model may be implemented directly within the DOTs, the headset, or a controller integrated into the headset, depending on the computational complexity involved.

Additionally, in certain aspects, the state transition model may incorporate quantifiable features, such as baseline and evoked features, derived from biophysical data. Non-invasive biophysical data, including but not limited to electrocardiography (ECG), electroencephalography (EEG), electromyography (EMG), electrodermal activity (EDA), electrooculography (EOG), functional near-infrared spectroscopy (fNIRS), and functional magnetic resonance imaging (fMRI), may be collected from the subject either prior to or during XCS therapy. Baseline features, calculated from the biophysical data in the absence of stimulus, may include EEG peak alpha frequency, EEG theta power, EEG broadband LFP, ECG baseline heart rate and variability, and fNIRS measures of brain oxygenation. Evoked features, derived from the biophysical data in response to stimuli (e.g., XCS, transcranial magnetic stimulation (TMS), or transcranial electrical stimulation (TES)), may include EEG peak evoked potential amplitude and timing, as well as EMG or EDA responses. Integrating these features may enhance the ability of the state transition model to map neurocardiac coupling and brain state from extracranial recordings, ultimately improving the precision of stimulation parameters.

In some aspects of the present disclosure, the triggered stimulation for dosing in the XCS therapy may comprise a single pulse or a sequence of pulses (e.g., a burst or a train), where pattern of these pulses e.g., amplitude or intensity of the pulses, a number of pulses, time intervals between pulses or bursts, and pulsewidth may vary depending on the specific therapeutic outcome or target engagement. In closed-loop neuromodulation, XCS therapy may encounter two dosing challenges when adjusting stimulation parameters: determining whether the stimulation dose is strong enough (e.g., in terms of amplitude or power) to activate the appropriate response, and whether the duration is adequate to achieve a therapeutic effect. The second challenge may lead to stimulation doses being either too high, imposing unnecessary burden, or too low, reducing efficacy. While many neuromodulation techniques address the first challenge by adjusting amplitude, XCS therapy uniquely excels at enabling the proper duration for a therapeutic response.

Experimental observations show that when pulses are delivered in bursts at a given amplitude, the number of pulses needed to elicit a neural response can vary across different days. Preclinical studies in a neural recruitment model demonstrated that, despite constant amplitude, the required pulse counts to activate a neural response fluctuates day-to-day. Some neuromodulation approaches adjust the amplitude of triggered stimulation based on the evoked neural response. However, this approach may be inefficient when the required pulse count varies to elicit the targeted neural response, as it may lead to longer times to find the correct parameters or excessive stimulation. To address this problem, an alternative approach is disclosed that includes delivering a stimulus burst at a fixed amplitude, then monitoring the neural response during the time intervals between pulses of the stimulation. When the targeted evoked response (e.g., SEP) is detected, the triggered stimulation may be terminated, indicating successful neural activation. This method improves efficiency by reducing stimulation time, lowers energy consumption, and reducing the risk of side effects by avoiding prolonged stimulation.

In some examples, stimulation may be delivered in trains of pulse sequences, where interburst intervals may be used to record neural responses and adjust stimulation accordingly. For example, a local field potential (LFP) may be measured during a first interburst interval yielding a power spectral density (e.g., showing increased low-frequency beta activity, 13-30 Hz). After one or more subsequent bursts, a second interburst interval may show a change in the power spectral density (e.g., reduced beta and increased gamma activity due to previous bursts of stimulation). The stimulation burst may continue to be delivered until such a change or shift is observed. Upon reaching the targeted shift or change, the stimulation may be terminated, alternatively, if no change occurs, stimulation parameters (e.g., pulse count, timing, or properties) may be adjusted. In some examples, stimulation-evoked potentials (SEPs) may replace LFPs for measurements.

Neural data during interburst intervals helps assess stimulation adequacy. If, for example, the neural data recorded during the interburst interval indicates that a sufficient dose has been delivered, therapy may be concluded early. The timing of future therapy sessions, as well as their expected duration, can be dynamically adjusted based on the results of the current session. For instance, if a morning session achieves neural measures suggesting a dose response within 75% of the expected duration, the system might either postpone an afternoon session to accommodate the stronger therapeutic effect or shorten the duration of the afternoon session by 25%. This dynamic adjustment may help the subject in efficient therapy scheduling based on their current response.

Closed-loop or human-in-loop neuromodulation techniques, involving externally powered electrical cortical stimulation (XCS), may provide dynamic control over stimulation parameters based on real-time neural feedback and (optionally integrated) biological data. These techniques may be configured to adjust stimulation parameters delivered by neuromodulation devices or implants (e.g., DOTs). The delivered XCS in an XCS therapy may be configured to treat different physiological conditions that may include, but are not limited to, neurological disorders (e.g., Parkinson's disease, epilepsy, Alzheimer's disease), psychiatric disorders (e.g., obsessive-compulsive disorder (OCD), depression, anxiety), movement disorders (e.g., essential tremor, dystonia, ataxia), cognitive decline disorders (e.g., mild cognitive impairment, dementia), chronic pain (e.g., fibromyalgia, back pain, neuropathic pain), and other conditions affecting brain function and motor control.

One application of these techniques is enhancing target engagement by dynamically adjusting stimulation parameters based on real-time neural and biological data. This feedback enables fine-tuning of the stimulation to achieve targeted therapeutic effects. Brain responses to the applied stimulation (e.g., XCS) are monitored in real-time, and based on this data, the stimulation parameters are adjusted via the controller to guide the brain through targeted transitions. These adjustments are informed by insights from the learned state transition model, optimizing the interaction between stimulation and neural activity. In this context, target engagement refers to modulating neuronal firing patterns, connectivity, and signaling pathways to improve the therapeutic effects of neuromodulation techniques. Target engagement refers to the process by which a stimulation—such as electrical, magnetic, or chemical—interacts with neural activity in the brain, modulating neuronal firing patterns, connectivity, or signaling pathways. Therefore, the disclosed neuromodulation techniques may be leveraged to improve target engagement to improve the interaction between the electrical brain stimulation (or XCS) and neural activity of the brain.

Maintaining target engagement may be challenging due to changes in the tissue environment around the neuromodulation device over time. Initially, the implant site is surrounded by saline, which is replaced by a protein-rich immune response and eventually encapsulation tissue that integrates with the skull. Each tissue stage has distinct electrical properties, which may alter the effectiveness of stimulation. Therefore, fine-tuned stimulation parameters shortly after implantation may become less effective as the tissue environment evolves. This tissue environment may continue to evolve over long periods as the subject ages or experiences other causes of neural degeneration. The disclosed techniques may address this issue by maintaining target engagement through continuously monitoring and adjusting the stimulation parameters associated with XCS to sustain a responsive or engaged brain state throughout the course of therapy. The techniques for maintaining target engagement are conceptually similar to those used for improving target engagement, with the primary distinction being the shift in objective—from enhancing engagement to enhancing its sustained maintenance over time.

Additional applications may include enhancing therapeutic outcomes by fine-tuning stimulation parameters to reduce side effects, such as drowsiness, nausea, discomfort or unwanted muscle contractions, based on ongoing neural responses. Further applications may include personalizing treatment by adapting stimulation parameters to the unique neurophysiological profile of each individual subject and continuously tracking the effectiveness of the therapy to improve long-term subject (or patient) outcomes.

In some aspects, the disclosed closed-loop neuromodulation techniques may also be used to prevent or mitigate relapses by dynamically modifying stimulation parameters in response to detected deviations from a targeted brain state, thus maintaining consistent therapeutic benefits. A relapse may refer to a recurrence or worsening of symptoms that the therapy was intended to address, such as return of a neurological or psychological condition after a period of improvement. The relapse risk may refer to a likelihood or a probability that the subject will experience a recurrence or worsening of symptoms following a neuromodulation therapy (or XCS therapy), typically after a predefined period of time. In cases where relapse risk is identified, closed-loop neuromodulation therapy (or XCS therapy) may be employed to help prevent relapse and maintain therapeutic benefits.

The process for predicting relapse risk may involve capturing both non-neural data (e.g., heart rate variability (HRV), galvanic skin response (GSR)) and neural recordings (e.g., local field potentials (LFPs)) from one or more implanted neuromodulation devices, such as DOTs, to monitor brain activity. Additionally, other biophysical data sources, including electroencephalography (EEG), functional magnetic resonance imaging (fMRI), and near-infrared spectroscopy (NIRS), may be employed to further assess neural activity and provide a comprehensive view of the subject's physiological and neurological status. Based on this multimodal data or (only) the neural recording, brain states e.g., including a healthy state, increasing risk state, relapse risk state, and a relapse or unhealthy state, may be estimated. While brain state estimation focuses on neural activity, disease state estimation may incorporate a broader range of data, including clinical assessments (e.g., HAM-D, MADRS) and voice recordings, to provide a detailed evaluation of both mental and physical symptoms of condition of the subject. This expanded approach to disease state estimation may be helpful as the relapse may not only manifest in changes to brain activity but also in shifts in psychological, emotional, and behavioral indicators, which may be captured through clinical assessments and voice recordings. For instance, changes in speech patterns (e.g., tone, fluency) may reveal early signs of relapse before neurological symptoms become apparent.

By integrating these diverse data sources, the disease state may be more accurately assessed, enabling an accurate prediction of the likelihood of relapses. When the disease state indicates a high risk of relapse, closed-loop XCS therapy may be triggered, adjusting stimulation parameters to stabilize brain activity and prevent further deterioration, thereby enabling continuous therapeutic effectiveness. Additionally, by continuously monitoring and analyzing the neural and non-neural inputs, the system may predict the likelihood of relapse and trigger appropriate therapy adjustments. For example, when the brain state remains within a healthy range, the system may stay in a monitoring mode. As the brain state approaches the threshold for relapse, the system may dynamically shift to a more proactive intervention, increasing monitoring and adjusting stimulation parameters, such as intensity and frequency, to stabilize the brain state. When a relapse risk is imminent, therapeutic intensity may be heightened, engaging both the neuromodulation devices and clinical support.

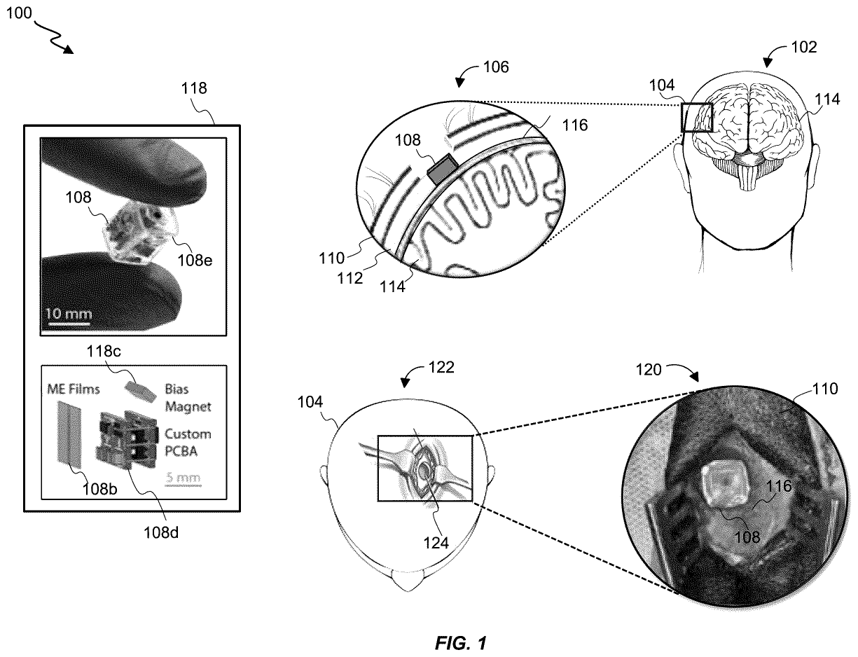

FIG. 1 illustrates an exemplary procedure 100 of implanting a neuromodulation device in accordance with some aspects of the present disclosure. Neuromodulation devices may be used to deliver controlled stimuli to targeted neural structures to regulate or alter neural activity. These devices may function through electrical, magnetic, or chemical means and are utilized in treating neurological conditions such as epilepsy, movement disorders, depression and chronic pain. Externally powered electrical cortical stimulation (XCS) therapy is one such invasive neuromodulation technique that may electrically stimulate a brain using epidural electrical cortical stimulations. In some aspects, a digitally programmable over brain therapeutics (DOT) 108 is employed as the neuromodulation device for epidural XCS. The DOT 108 may include stimulation electrodes, configured to deliver precise electrical impulses (or stimulations) to a targeted cortical region without penetrating the dura mater 116. The DOT 108 is dynamically programmable, enabling dynamic adjustment of stimulation parameters based on subject response in real-time.

In particular, FIG. 1 depicts the implantation procedure 100 of the DOT 108 in which the neuromodulation device e.g., DOT 108 is placed epidurally, resting above the dura mater 116 without direct contact with the brain 114. FIG. 1 illustrates multiple views and zoomed-in perspectives of the implantation procedure 100. For example, a general location of an implantation site 104 is marked at a predefined cortical region in a front view of the head 102. The front view 102 may provide an anterior perspective, helping to orient the positioning of the neuromodulation device relative to cranial landmarks. Once the cortical region is identified, the neuromodulation device-DOT 108, including stimulation electrodes may be surgically implanted into the target region through a burr hole procedure. In the burr hole procedure, a small (burr) hole 124 may be drilled into the skull of the subject, typically at a location that provides direct access to the cortical region of the brain 114.

A more detailed top view of the head 122 illustrates a precise placement of DOT 108. The top view 122 may highlight another or a same target cortical area and the positioning of the burr hole 124, which is drilled through the skull 112 for accessing the dura mater 116. In a zoomed-in cross-sectional view 106, a layered cranial structure is demonstrated, detailing how the skin 110 is incised and retracted to expose the skull 112 and DOT placement above the dura mater 116, enabling protection for the brain 114. Following the implantation of DOT 108, the skin 110 may then be repositioned and sutured, enclosing DOT 108 within the epidural space. For illustrative purposes, FIG. 120 shows the implantation of the DOT and skin suturing in a porcine model. Once the recovery phase is complete, the stimulation electrode associated with the invasive technique may become handy for ongoing monitoring and stimulation with different adjustments made as necessary based on the biological data or the neural data of the subject. Additionally, FIG. 1 depicts a general view of DOT 118, including the stimulation electrodes (not shown), magnetoelectric film 108b, bias magnet 108c, custom designed printed circuit board (PCB) 108d enclosed in an encapsulation material 108e that comprises a biocompatible layer for long-term stability and protection against biological interference.

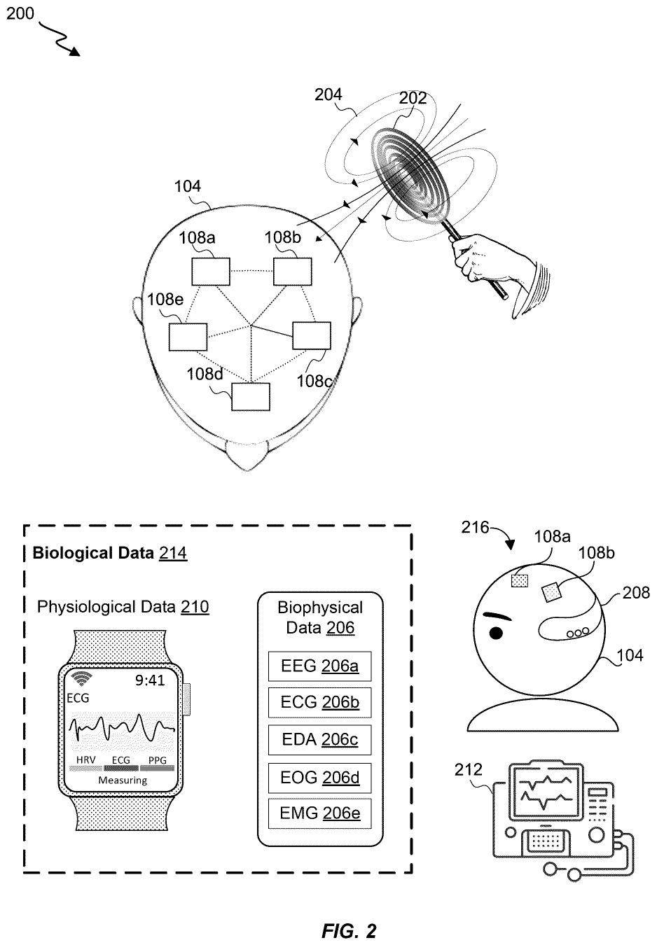

FIG. 2 depicts an illustration of a neuromodulation system 200 comprising a set of neuromodulation devices implanted in a head of a subject, in accordance with some aspect of the present disclosure. In some aspects, a network of DOTs (e.g., 108a, 108b, 108c, 108d and 108e) may be strategically implanted within the skull 112, over different cortical regions, thereby creating a brain mesh or a neural mesh of DOT nodes. The network of DOTs may form a distributed system capable of interacting in real-time, where each DOT may perform a variety of functions, including stimulating the brain 114 by delivering the XCS, recording neural activity via collecting neural data, communicating with neighboring DOTs, and executing local computations to fine-tune stimulation parameters based on real-time data. The neuromodulation system 200 may further include a magnetic coil system configured to power and/or program the neuromodulation devices. Specifically, a single magnetic coil 202, or an array of magnetic coils, may be employed to wirelessly deliver both power and digital inputs to the DOTs. These magnetic coils may be integrated into a headset that serves as an interface for power transmission and input delivery to the neuromodulation devices.

In some configurations, the headset may operate autonomously or may be augmented with a software controller. The software controller may provide dynamic user inputs for the adjustment of stimulation and recording parameters. Such inputs may either be pre-programmed or manually controlled by the user. In some implementations, one or more of the DOTs may be equipped with small power reserves, enabling these devices to continue performing specific functions, such as neural recording, even in the absence of external power supplied by the headset. These power reserves may help the DOTs to maintain functionality during brief periods of disconnection from the external power source. The DOTs may be capable of stimulating the brain 114 using electric currents and recording neural activity, such as local field potentials (LFPs) and evoked potentials. Each DOT in the brain mesh may be configured to communicate with other DOTs through radio frequency (RF) protocols. In addition to direct communication between the DOTs, indirect communication may be employed via the headset or a controller with the headset, which acts as the central hub for powering and controlling the entire neuromodulation system comprising magnetic coils and neuromodulation devices.

In some instances, a DOT (e.g., 108a) may monitor the stimulation activity of a neighboring DOT (e.g., 108b) and, based on this observation, trigger its own operation. For example, the observed stimulation activity may prompt the first DOT to initiate recording functions or to adjust its stimulation output. This inter-DOT communication may create a synergistic network effect, enhancing the overall adaptability of the neuromodulation system. Furthermore, the DOTs may be capable of performing local computations to fine-tune the stimulation parameters. This process may involve updating stimulation settings for each DOT based on its own neural recordings or the neural data, as well as those from other DOTs that are part of the relevant brain mesh. These neural recordings may be processed to derive brain state estimates or neural signatures, which correspond to the mental state or a therapeutic state of the subject e.g., specifically indicating whether a prior treatment maintains its efficacy over a defined period, as quantified by measurable physiological or neural response thresholds. The derived brain states may be computed using recordings from either a single DOT or from a subset of DOTs in the network, depending on the specific application. This cross DOT communication may be used to deliver phase locked stimulation, such that the stimulation of one DOT leads or follows in a specified phase relationship the stimulation of another DOT or, more likely the neural responses caused by another DOT. As an example, a first DOT may create a stimulus response in the beta frequencies (13-30 Hz) and a second DOT may entrain a higher frequency stimulus (gamma at 50-70 Hz) following the first response, mirroring a pattern known to encode information between brain regions in healthy functioning neural systems.

While a complete mapping of brain state typically requires extensive recording from number of nodes in the brain network, the inherent recurrent loops in brain networks help in a more efficient approach. Specifically, recordings from a sparse subset of nodes may often provide sufficient information to infer the overall brain state. The present disclosure leverages this phenomenon, enabling the DOTs to generate accurate brain state estimates by recording from a strategically selected subset of nodes, rather than requiring a full set of brain network recordings. In one implementation, RF protocols serve as the communication backbone for the DOTs, enabling synchronized operation of the system. RF communication may enable real-time data exchange between the DOTs, as well as communication with the central control unit (e.g., the headset or software controller), which directs the neuromodulation therapy based on the ongoing neural activity and the parameters of the system.

In further embodiments, the neuromodulation system 200 may be enhanced by integrating biological data 214 associated with a subject 216 from other sensors. The biological data 206 may include e.g., conventional electroencephalography (EEG) 206a, EEG embedded in the headset 208, electrocardiography (ECG) 206b, electrodermal activity (EDA) 206c, electrooculography (EOG) 206d, electromyography (EMG) 206e, and physiological data 210 from one or more physiological sensors. The physiological sensors e.g., wearable sensors included in a wrist-worn device, may continuously monitor the physiological data 210 of the subject 216 in real time, thereby providing complementary data that may be incorporated into the neuromodulation system 200. The physiological sensors may include, but are not limited to, heart rate sensors to monitor cardiovascular activities, electrodermal sensors to track skin conductance and stress levels, temperature sensors to record body temperature, motion sensors (e.g., accelerometers and gyroscopes) to detect physical activity and movement patterns, blood oxygen sensors to measure oxygen saturation (SpO2) levels, and ECG sensors to monitor heart rhythm and its electrical activities. The biological data 214 collected by these sensors may be transmitted (e.g., wirelessly or with wires) to the headset 208 or a computing device 212, which may adjust the XCS therapy based on real-time changes in the biological data 214 or the neural data of the subject.

In addition to biological data monitoring, the neuromodulation system 200 may also integrate audio devices (e.g., microphones, or speakers) to continuously record voice logs of subject 216 that may provide insights into the mental state or disease state of the subject. The neuromodulation system 200 may also occasionally present a questionnaire (e.g., for clinical assessments) to the subject 216 via a graphical user interface (GUI) such as embedded within the computing device 212. The questionnaire may be designed to predict the mental state or the disease state of the subject 216 based on the responses from the subject 216. The voice logs and questionnaires may be used by the computing device 212 for further analysis, assisting in decisions about the XCS therapy to be delivered to the subject 216. In some aspects, the subject 216 may be notified when to wear the headset 208 for administering the XCS therapy. Such notifications may enable that subject 216 to receive timely and appropriate treatment based on the subject's current physiological and mental condition.

The additional biological data 214 (or signals) may offer further insights into physiological and cognitive states of the subject 216, thereby enabling more precise brain state (and/or disease state) estimation and personalized neuromodulation therapy. The integration of these diverse sensor inputs into the controlling algorithms may help the neuromodulation system 200 to improve the neuromodulation process based not only on direct neural recordings but also on biological data 214. This holistic approach may facilitate more accurate detection of brain states and mental states, improving the efficacy of neuromodulation therapy.

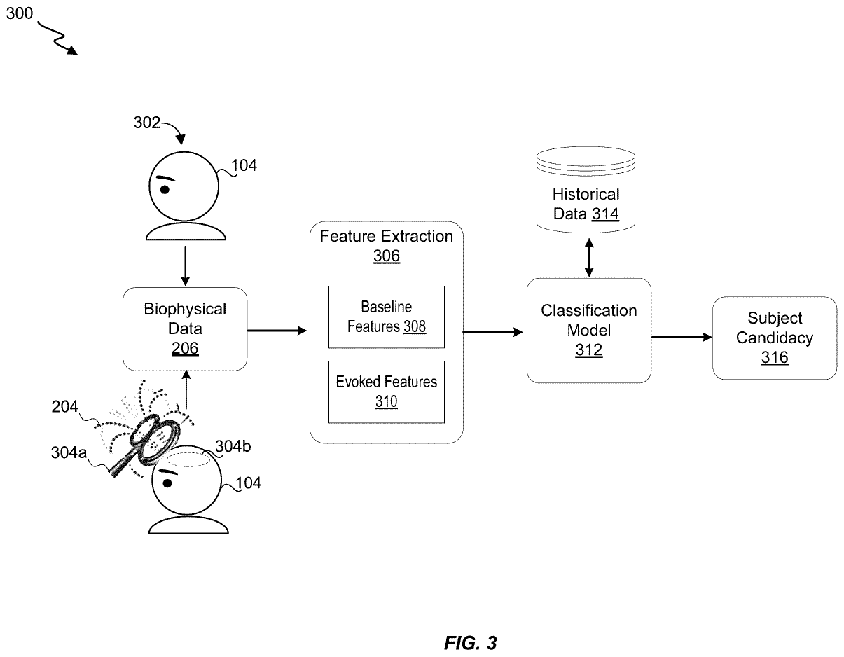

FIG. 3 illustrates a block diagram 300 for predicting subject candidacy 316, in accordance with some aspects of the present disclosure. In some aspects, the disclosed techniques may include prediction of candidacy of the subject 216 for the externally powered electrical cortical stimulation (XCS) therapy. This prediction process may contribute to efficiency of XCS therapy, which is configured to modulate brain activity by delivering electrical stimulation to the cortex. The goal of such prediction may be to identify subjects who are most likely to benefit from XCS therapy, based on their unique neural and biological characteristics, before the implantation of XCS devices (or neuromodulation devices). The disclosed prediction techniques of candidacy for XCS may include analyzing a variety of biophysical signals or (biophysical data 206) and performing feature extraction 306 to correlate with symptom relief and therapeutic outcomes following XCS activation. Feature extraction 306 may involve deriving meaningful and quantifiable features (such as frequency bands, amplitudes, and timings) from raw biophysical data 206 (e.g., EEG, ECG, etc.) that may be used for further analysis or classification, such as predicting subject candidacy 316 for XCS therapy. The extracted features may help assess the connectivity state of the brain—the degree to which different cortical regions communicate and synchronize with one another.

The biophysical data 206 may be acquired non-invasively from the subject prior to XCS device implantation or XCS therapy for providing valuable insights into subject responsiveness to XCS based on comparisons (e.g., pre therapy and post therapy). The extracted features from biophysical data 206 to predict subject candidacy 316 for XCS may include baseline features 308 that comprise specific metrics derived from spontaneous brain activity in the absence of an external stimulus such as EEG rhythms or oscillations (alpha waves, theta waves detected via EEG) or physiological features such as heart rate variability. These brain activities may reflect the brain's natural response, baseline neural patterns and may offer valuable insights into overall brain function and brain connectivity—the way different parts of the brain are communicating with each other naturally. By analyzing these natural brain rhythms, brain state determination 404 may be performed to evaluate whether the brain 114 is in a state that would be conducive to responding well to XCS.

The baseline features 308 may include e.g., EEG peak alpha frequency, EEG theta power, EEG broadband local field potential (LPF) and/or ECG baseline heart rate and heart rate variability. For example, EEG peak alpha frequency measures a dominant frequency of brainwave oscillations in the alpha band (8-12 Hz), which is associated with relaxed, wakeful states. Variations in alpha frequency may reflect brain network connectivity of subject 216 and responsiveness to cortical stimulations. Similarly, theta rhythms (4-8 Hz), linked to relaxation, creativity, and working memory, may indicate abnormal brain functioning or resilience to interventions such as XCS. EEG broadband LFP may reflect an overall electrical activity of neurons within a given cortical area, offering a detailed representation of the brain's electrical dynamics. Changes in EEG broadband LFP may suggest dysfunctions or alterations in cortical networks. ECG baseline heart rate and heart rate variability measurements may provide insights into autonomic nervous system functioning, which is intrinsically linked to neural regulation and may affect the efficacy of XCS interventions. Heart rate variability, in particular, is a useful measure of physiological flexibility and stress resilience, and can be a modulated downstream by cranial stimulation in certain network targets such as the dorsal lateral prefrontal cortex.

In addition to baseline features 308, evoked features 310 may be measured in response to external stimuli such as transcranial magnetic stimulation (TMS) or transcranial electrical stimulation (tES), including transcranial AC (tACS) and DC (tDCS) stimulation. For example, EEG peak evoked potential amplitude and EEG peak evoked potential timing may provide critical information about how the brain responds to external stimulation, which may further inform predictions regarding XCS response. EEG peak evoked potential amplitudes are the peaks of neural responses to an external stimulus, often used to assess sensory processing and cortical excitability. External stimulus such as TMS or TES may be used to apply a non-invasive electrical or magnetic field to the brain 114 to modulate neural activity. TMS uses a TMS coil 304a to generate a rapidly changing magnetic field 204 that induces an electrical current 304b in the underlying brain tissue, enabling assessment of cortical excitability and connectivity by measuring evoked potentials or neural responses.

TES, on the other hand, involves applying a weak electrical current to the scalp (or skin 110) through electrodes, directly stimulating the cortex to affect neural activity, with the resulting changes in brain function used to gauge brain responsiveness and predict outcomes for neuromodulation therapies. Both techniques may help measure response of the brain 114 to stimulation, providing valuable data for assessing candidate suitability for treatments such as XCS therapy. Since both XCS and TMS target the dorsolateral prefrontal cortex (DLPFC), responses to TMS may provide important predictive information regarding how well the subject may respond to XCS therapy. Higher or lower amplitudes may indicate different brain states or levels of connectivity in response to stimulation.

The extracted features from the biophysical data 206 may then be input into a classification model 312, which uses a combination of historical data 314 from past TMS and XCS subjects to predict how likely a new subject is to benefit from XCS therapy. The classification model 312 may analyze patterns across various signal types and features, such as EEG metrics, heart rate variability, and TMS responses, comparing them to known outcomes from previous subjects who have undergone TMS or XCS treatments. By incorporating data from both therapies, the classification model 312 may capitalize on the shared therapeutic target (the DLPFC), enhancing its predictive accuracy. Each feature in the model, such as EEG-derived metrics or TMS response, may be loosely correlated with the subject candidacy 316 for XCS. When combined, these multiple features form a stronger prediction model. The system takes advantage of this multi-feature approach, to distinguish between subjects who are likely to benefit from XCS and those who may not. Further enhancing the classifier, machine-learning techniques may help improve continuously, evolving as new data from subjects may be collected over time. This predictive model may thus serve as a powerful tool to guide clinical decision-making XCS therapy applicable to those most likely to experience significant therapeutic benefits.

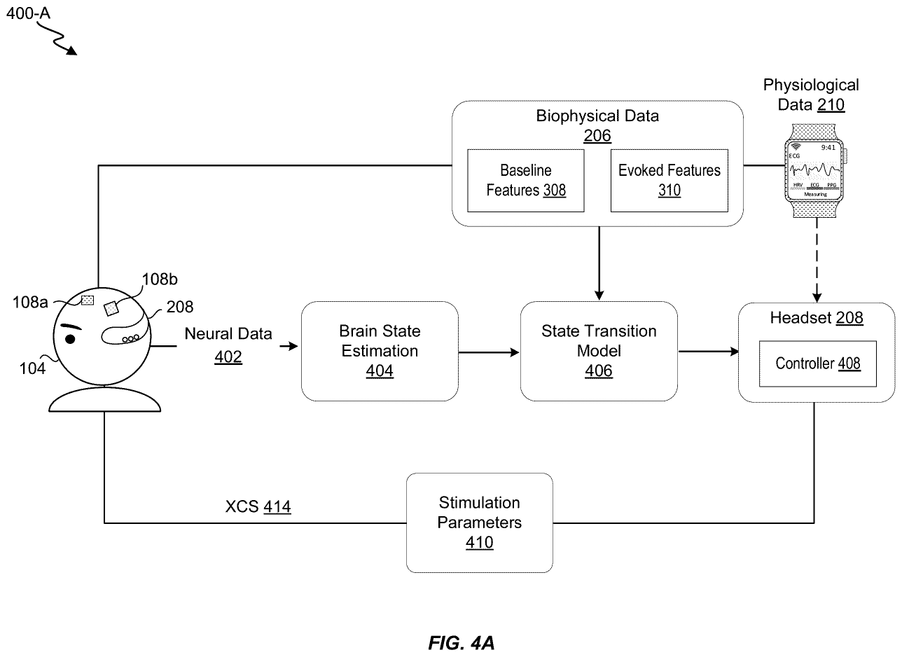

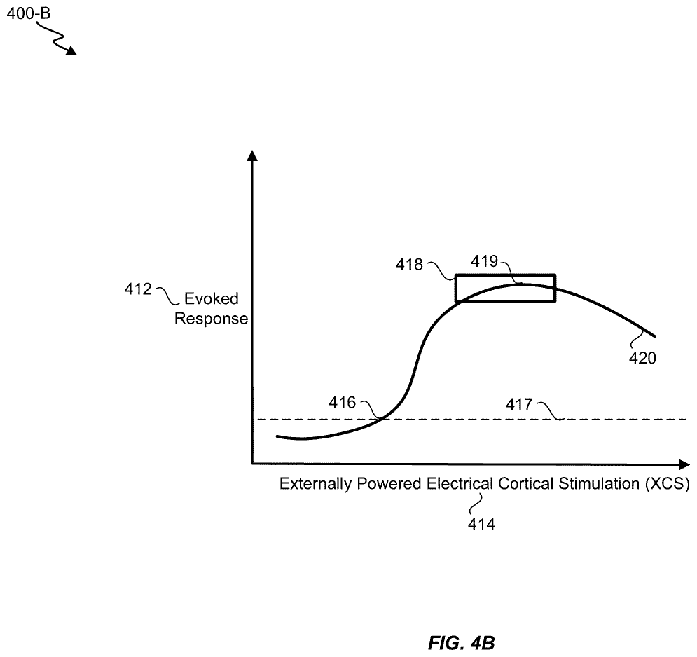

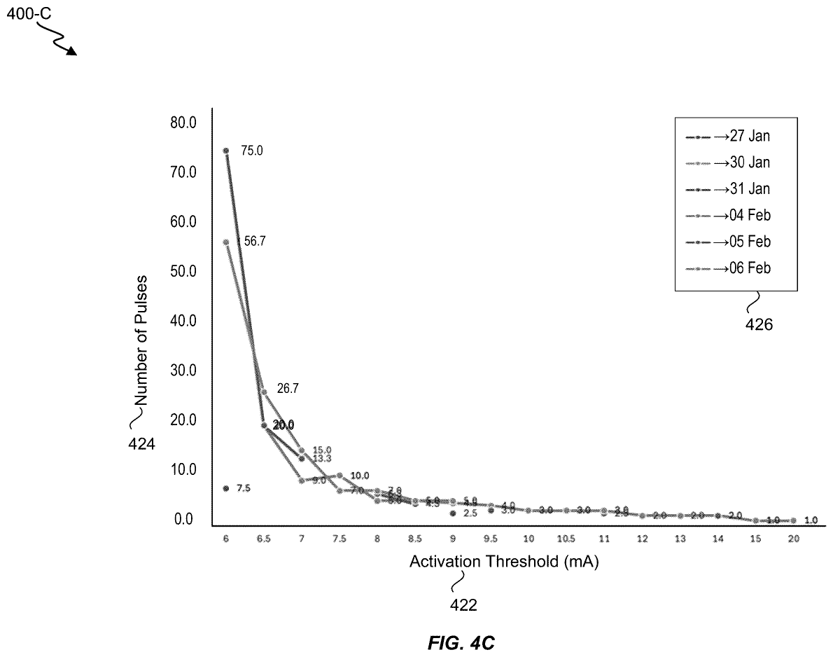

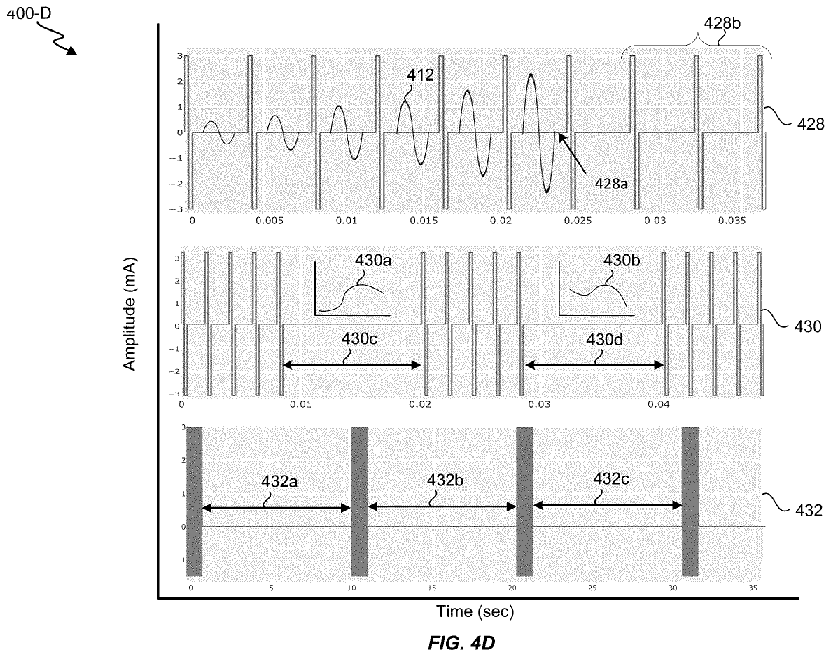

FIG. 4A depicts an exemplary illustration 400-A for providing closed-loop or “human-in-the-loop” electrical stimulation aimed at enhancing target engagement for XCS therapy, in accordance with some aspects of the present disclosure. Stimulation of the brain may activate a population of neurons, where changing the parameters of stimulation (current amplitude, pulse width, frequency, to name a few) may alter the extent of neural activation with stimulation. For effective XCS therapy, an adequate amount of neural activation within a target region of cortex may be applied. Additionally, neurons in downstream regions of the brain, which are connected through neural pathways, may also be activated. This activation may happen through synaptic release mechanisms, where stimulation of one neuron causes it to release neurotransmitters that activate neighboring neurons. The neighboring neurons may include those located further along the communication pathways of the brain 114 (e.g., downstream region), which provides the full therapeutic benefit of the stimulation. In some aspects, the present disclosure introduces closed-loop recording and stimulation techniques that record neural data 402 from one or more DOTs e.g., 108a, . . . , 108n within a brain network, map the brain state, compute the suitable stimulation parameters, and subsequently reprogram the DOTs to deliver the appropriate XCS for improved target engagement.

In some examples, externally applied electrical cortical stimulation (XCS) or stimulation may comprise a single pulse or a sequence of pulses (e.g., a burst or a train), where amplitude or intensity of the pulses, a number of pulses, time intervals between pulses, and pulsewidth. The pattern of these stimulations may vary depending on the specific therapeutic outcome or target engagement. Neural data 402, capturing neural activity, may then be recorded continuously or at specified intervals, such as during the interburst intervals between stimulation sequences, over extended periods of time from one or more DOTs implanted in cortical regions. A cortical region refers to a particular area of the brain's outer layer, the cortex, responsible for higher-level functions e.g., sensory processing, motor control, and cognition. The neural data 402 may include local field potentials (LFPs), stimulation-evoked potentials (SEPs), or event-related potentials (ERPs), obtained at one or more DOTs before, during, and/or after therapeutic stimulation delivery. These neural data 402 may represent time-series data, as these recordings track the electrical activity of the brain over time.

LFPs may reflect the combined electrical activity of large groups of neurons within a local region of the cortex, recorded continuously as fluctuations in voltage over time. The local region may refer to a smaller, specific area within the broader cortical region, typically in proximity to the DOT electrode, capturing the synchronized neural activity of neurons in that area. SEPs may refer to the brain's electrical response to a specific external stimulus (e.g., comprising a stimulation pulse, a series of stimulation pulses in a burst, or a short series of bursts of stimulation pulses) that may be recorded over time after the stimulus is applied, capturing both immediate and delayed neural responses. ERPs, similarly, are time-locked brain responses to an event (e.g., a sensory or cognitive task or therapy, physiological triggers), and are calculated by averaging the brain's electrical responses to repeated stimuli to enhance the signal of interest.

The neural data 402, often represented as time-series data that captures the dynamic electrical activity of the brain over time, may be processed to derive estimates of brain state (e.g., engaged, relaxed, or a particular functional mode) by applying various computational techniques. These neural data 402 may provide real-time information about the brain activity as well as its current state. The techniques, such as recruitment curve mapping for evoked potentials and principal component decomposition of local field potentials, may be employed to simplify the complex, high-dimensional neural data 402 into more interpretable forms that reveal underlying neural patterns. Recruitment curve mapping may be used to estimate brain states by analyzing how different regions of the brain respond to various levels of stimulation. This technique may help in identifying a threshold at which neural activation begins, offering insight into the brain current level of engagement or alertness. In this technique, different levels of stimulation intensity may be applied to cortical regions via DOTs, and the brain response to each level of stimulation is recorded as time-series data (e.g., SEPs).

The recorded responses may then be plotted against the stimulation levels to generate a recruitment curve, illustrating how neural activation increases as a function of stimulation intensities or the design of a stimulation burst or pattern. The steepness and shape of the recruitment curve may indicate a brain state that may be classified as a responsive state (indicating active engagement or alertness) or a non-responsive state (indicating reduced engagement or inactivity). The brain state of responsiveness may vary depending on stimulation waveform design for targeted neural response. For example, in some applications, a lower threshold and more pronounced activation suggest an alert or engaged state, while a higher threshold and less activation may indicate a more relaxed or less engaged state. These variations in brain state receptivity can change over time for various reasons, including shifts in neural engagement and anatomical differences. Alternately, specific stimulation waveforms may recruit neural activation more or less easily, regardless of brain state.

For example, if a subject is very focused or alert, the brain may respond quickly (at a low threshold) to external stimulus. The associated recruitment curve may show a sharp increase in neural activation as soon as a low level of stimulation is applied, suggesting a brain state that corresponds to high engagement and responsiveness. Similarly, if a subject is in a relaxed or drowsy state, or is dosed with certain medications that change the excitability of brain networks, the brain may not respond until a stronger stimulus (higher intensity) is applied. Additionally, anatomical considerations, such as a posture or movements of the subject, may displace an electrode closer or farther from neural tissue, requiring stronger stimulus to yield similar activation levels. The corresponding recruitment curve may show a gradual or delayed increase in activation, indicating that the brain state corresponds to a non-responsive or non-engaged state (or a lower responsive, lower engaged state, or an anatomically less sensitive state). This technique may provide a quantitative approach to understanding the dynamic responses of the brain to external stimuli, helping to estimate its functional state in real time. Principal component decomposition may reduce the complexity of LFP data by identifying the most significant components that explain the variance in neural activity, thereby isolating the primary neural dynamics relevant for brain state estimation 404.

In some implementation, including a single DOT, the brain state may be estimated based on the neural responses to stimulation-evoked potentials (SEPs), which are a direct reflection of the brain's activity following external stimulation. This estimate may incorporate the reverberation of neural activity, often referred to as evoked resonant neural activity or local evoked potentials, which may be caused by the recurrent synaptic connections in the brain. These connections may lead to a sustained oscillation of neural activity, providing valuable insight into the brain's response to stimulation. In other implementations, including multiple DOTs, the neural activity may be recorded from several nodes distributed across the brain network. This expanded set of data may enable a more detailed estimation of the brain state, as it captures the interactions between multiple regions of the brain, providing a holistic view of the brain's activity. Through the use of these multiple recordings, a more accurate and refined brain state model may be computed, enabling better-informed decisions regarding suitable stimulation parameters for therapeutic purposes.

Additional techniques, such as spectral analysis or power spectral density (PSD) analysis of the neural recordings or the use of machine-learning techniques, may further enhance the accuracy of brain state estimation 404. Spectral analysis may assist in the examination of neural oscillations across different frequency bands, which may provide further insight into the dynamic state of the brain. For example, power spectral density (PSD) analysis may be used to examine the frequency components of entire LFP data or within specific frequency bands to understand underlying neural oscillations that contribute to brain state dynamics. Meanwhile, machine-learning techniques, including clustering, classification and regression models, may be employed to predict brain states based on historical data, improving the adaptability and precision of the stimulation protocols. For example, the neural recordings from one or more brain regions may be clustered into distinct brain states. The clustering may be performed based on metrics such as principal component analysis (PCA) or other advanced signal decomposition techniques. These techniques may enable the extraction or generation of features from the neural recordings, thereby identifying patterns associated with different mental or cognitive states. The neural data 402 may be utilized to identify specific brain states and employ stimulation protocols to facilitate transitions between these states, thereby improving brain function.

Once brain states are determined, the stimulations may be applied throughout the recording period for active perturbation of brain activity to promote transitions between different states. This process may be driven by a state transition model 406, such as a Hidden Markov Model (HMM), to learn how the brain transitions from one state to another over time based on the ongoing neural activity. This model may use data from previous brain states and stimulation outcomes to understand how different stimulation parameters 410 (e.g., intensity, pulse width, frequency, number of pulses delivered, pattern of pulses delivered) affect brain state transitions. The model may learn the dynamic changes between brain states, both with and without the application of stimulation. The state transition model 406 may enable adaptive learning, where the stimulation patterns 410 may be fine-tuned over time to guide the brain 114 through targeted transitions. The state transition model 406 may be implemented either directly within the DOTs or within the headset 208, or alternatively, within a controller 408 integrated into the headset 208, at either the hardware or software level. The choice to implement the state transition model 406 at DOT or at the controller 408 may depend on the complexity of the computations involved.

While some computations, such as those related to local brain activity, may be performed directly on the DOTs, others, which may require more computational power, may be offloaded to the headset 208 (or the controller 408). Additionally, in some aspects, the state transition model 406 may integrate baseline features 308 such as heart rate, EEG local field potential along with the evoked features 310 such as EEG evoked potential derived from the biophysical data 206. This integration may enhance the ability of the state transition model 406 to map neurocardiac coupling and brain state from extracranial recordings, both of which may be incorporated into the state transition model 406 to improve target engagement and the precision of stimulation parameters 410.

Based on the learned state transitional model, stimulation parameters 410 may be determined and continuously adjusted in real-time to enhance target engagement —that is, to stimulate the brain in a way that improves therapeutic effectiveness. If the brain moves into a state that is not ideal for stimulation, the stimulation parameters 410 (e.g., frequency or intensity) may be automatically adjusted to bring the brain into a more favorable state, facilitating better neural engagement. The exemplary system 400-A may continuously monitor the neural data 402, estimate brain state, make predictions about future states by leveraging the state transition model 406, and adjust stimulation in real-time based on this ongoing feedback. These closed-loop control techniques at both the DOT level and the controller level may help the neuromodulation techniques to adapt optimally based on real-time brain activity and dynamic neural state changes.

As mentioned earlier, the DOTs may communicate with each other using radio frequency (RF) protocols, which enables exchanging neural data and coordinate stimulation efforts in real time. Some computations, particularly those related to network engagement, may be better suited for processing at the controller level (e.g., within the headset 208), where data from multiple DOTs may be aggregated and analyzed. For example, while target engagement—measured by the response of a DOT to its own stimulation—may be efficiently processed locally at the individual DOT, network engagement, which reflects how stimulation in one DOT may affect other areas of the brain 114, may be best analyzed with access to the full network of recordings from all DOTs. The controller 408 may have a global view of the neural data and activities performed by neuromodulation system, enabling it to fine-tune stimulation parameters 410 for each DOT based on interactions across the entire brain network. This approach may assist the neuromodulation system to not only address local neural activity but also maintain coherence across the network, facilitating broader therapeutic effects.

FIG. 4B illustrates an exemplary curve 400-B demonstrating a closed-loop neuromodulation technique that dynamically adjusts stimulation parameters 410 based on the real-time neural data 402, in accordance with some aspects of the present disclosure. The x-axis represents external powered electrical cortical stimulation (XCS) 415 (or a stimulation dose), which corresponds to associated stimulation parameters e.g., amplitude, pulse width, frequency, number of pulses, and/or duration of bursts, while the y-axis represents the corresponding evoked (neural) response 412. The neural data 402 represents continuous measurements of the brain's electrical activity, such as local field potentials (LFPs) or stimulation-evoked potentials (SEPs). These signals may capture the brain response to both baseline activities and external stimuli (i.e., neuromodulation). Specifically, the evoked response 412 may reflect the neural activity triggered by the application of stimulation parameters.

In some examples, the exemplary curve 400-B may imply a recruitment mapping curve where different intensities of stimulations or other measures of stimulation dose, including the number of pulses of stimulation delivered, the number of bursts of stimulation delivered, or the timing of the pulses or the bursts are plotted against the collected neural responses. Initially, increasing the stimulation does not have significant changes in evoked responses 412, below a threshold 417. It may be observed from the curve 400-B that as the stimulation dose increases (moving along the x-axis), the evoked response 412 (plotted on the y-axis) rises sharply above the threshold 417, reflecting enhanced neural engagement stimulation.

The state transition model 406 may predict how the brain state will evolve based on the evoked response 412. The model may forecast when the evoked response 412 will reach its peak evoked response 419 (or engagement level), as illustrated in the exemplary curve 400-B within an enclosure 418. At this point, the brain has reached an optimal state of stimulation, where the neural response is maximized. The state transition model 406 is trained to determine when further increases in stimulation may result in diminishing returns. Beyond the peak engagement point (e.g., at 419), the curve 400-B shows that further increases in stimulation (whether through higher amplitude, longer pulse duration, etc.) lead to a decline in the evoked response 412. This drop may be due to phenomena such as neural adaptation or desensitization, where the brain becomes less responsive to continued stimulation. The state transition model 406 may use this feedback to dynamically adjust the stimulation parameters 410, enabling that the system never over-stimulates the brain and keeps it within the responsive and therapeutic state. This dynamic adjustment based on real-time feedback from the neural data 402 may create the closed-loop system, where the XCS 414 is fine-tuned continuously or at specified intervals for improved brain engagement at all times.

In closed-loop neuromodulation technique, there may be two dosing challenges associated with XCS therapy, while adjusting the stimulation parameters. The first challenge is determining whether the stimulation dose is sufficiently intense (e.g., in amplitude) to reliably engage or activate the appropriate response mechanism. The second challenge pertains to whether the duration of the stimulation is adequate to deliver a therapeutic dose. A consequence of the second challenge may be that, in an effort to exercise caution, many subjects may receive a dose that exceeds what is necessary, thereby imposing an unnecessary burden on the subjects. Conversely, other subjects may receive an insufficient dose, leading to a lack of therapeutic efficacy. Many systems use neural measures to address the first challenge e.g., by continuously adjusting amplitude to maintain the appropriate response mechanism. The disclosed closed-loop XCS therapy may provide a unique advantage in addressing the second challenge. Specifically, XCS therapy excels in determining whether a sufficient dose has been delivered to achieve a therapeutic response.

In particular, when bursts of pulses are delivered in a sequence at a given amplitude, experimental observations have revealed that, on different days, there is a variability in the number of pulses required to elicit a neural response. This observation arises from preclinical studies conducted in a model of neural recruitment, where the amplitude required to achieve neural recruitment was measured across varying numbers of stimulus pulses on different experimental days. These studies demonstrate that the precise number of pulses needed to activate a neural response can fluctuate day-to-day, even when the amplitude remains constant.

FIG. 4C illustrates an exemplary graph 400-C depicting a relationship between activation thresholds 422 to the number of pulses 424 required to elicit a neural response across multiple experimental sessions, in accordance with some aspects of the present disclosure. The activation thresholds 422 (measured in milli amperes, mA) are drawn at the x-axis, while the y-axis represents the number of pulses 424 delivered. In this exemplary graph 400-C, different colored lines correspond to separate experimental sessions that were conducted on separate days, as indicated in the legend 426. The data show that at lower activation thresholds (e.g., approximately around 6 mA to 7 mA), a significantly higher number of pulses is required to elicit a neural response. This number sharply decreases as the activation threshold increases, demonstrating a non-linear trend. Additionally, there is a day-to-day variability in the number of pulses 424 required at similar activation thresholds, highlighting fluctuation in neural recruitment across experimental sessions.

Some of the predicate systems may utilize an evoked response to titrate the amplitude of a continuous stimulation waveform, adjusting (only) the amplitude based on the neural response observed. However, this approach may not be efficient in situations where the number of pulses of the applied stimulation required to elicit a response varies. This is because, if a system continuously adjusts the amplitude based on the evoked response, this may lead to inefficiency because it may take longer to find the correct stimulation parameters or result in excessive stimulation. In contrast, based on the experimental observation of FIG. 4C, an alternative approach is disclosed: delivering a stimulus burst at a predefined and fixed amplitude rather than continuously adjusting the intensity of the pulses. Then, the system monitors the neural response, and when it detects an evoked response (such as a stimulus-evoked potential (SEP)), it will terminate the stimulation, indicating that neural activation has been achieved. This approach may offer several advantages. First, it may improve the efficiency of stimulation, as less time is required to achieve a neural event. Second, it may reduce energy consumption by limiting the duration of unnecessary stimulation. Third, this method may likely reduce the risk of stimulation-related side effects, as it avoids prolonged or excessive stimulation.

FIG. 4D shows illustrative examples of stimulation bursts, each comprising sequence of pulses, in accordance with some aspects of the present disclosure. The stimulation burst 428 comprises a sequence of ten pulses, where each pulse of the stimulation burst is delivered with a predefined and fixed amplitude. As depicted, the evoked response 412, represented as a sine wave, gradually increases in amplitude with successive pulses. At 428a, the evoked response 412 reaches a predetermined threshold, indicating that neural activation has been successfully achieved. Upon detection of this threshold, further stimulation may be terminated, thereby omitting the remaining pulses in the stimulation burst (as indicated by the region 428b). By dynamically ceasing stimulation upon achieving neural activation, this approach enhances energy efficiency by reducing the total stimulation time required to elicit a response, and mitigate potential adverse effects associated with excessive or prolonged stimulation. Additionally, it reduces power consumption by preventing unnecessary pulses, thus extending the operational life of implantable devices.

Alternatively, stimulation may be delivered in trains of pulse sequences, as illustrated by the stimulation burst 430 of FIG. 4D. The interburst intervals between each pulse sequence e.g., 430c and 430d may be used to record neural data 402 and determine how or whether to continue delivering the stimulation. Following this approach, neural data may be recorded during a first interburst interval 430c to establish a first measure-a local field potential (LFP) processed to yield a power spectral density 430a. For example, showing an increased low-frequency activity such as in the beta frequency range of 13-30 Hz. The power spectral density (430a) shows the distribution of brain activity across different frequencies. After one or more subsequent bursts, a different power spectral density 430b may be observed during a second interburst interval such as 430d as a result of the preceding bursts. For example, the data collected during the second interburst interval 430d may show a change, such as a reduced beta activity and increased gamma activity, due to the previous bursts of stimulation. The stimulation burst 430, representing a train of bursts, may continue to be delivered until such a change or shift is observed, at which point the stimulation may be terminated. If no change occurs, the stimulation parameters e.g., the number of pulses in a burst, the timing between bursts, or the properties of each pulse (pulse width, amplitude and/or frequency) may be adjusted to improve the effect or reach the targeted neural response. In some other examples, Alternatively, the first measure (derived from the neural data during the first interburst interval 430c) and the second measure (derived from the neural data during the second interburst interval 430d) may represent stimulation evoked potentials (SEPs) (instead of LFPs).

The stimulation waveforms e.g., 428 and 430 intended for XCS involve the delivery of trains of pulses with a defined length, which may be terminated upon detection of targeted engagement. These pulse trains may be delivered in longer sequences, which are themselves administered repetitively or intermittently over extended periods, potentially lasting up to 10 minutes or more. Neural data or recordings obtained during the intervals between these extended bursts may be utilized to evaluate the adequacy of the stimulation dose administered during the session. Upon reaching a targeted therapeutic dose, the session may be terminated early, thereby enhancing convenience for the subject or patient. Alternatively, if the targeted dose is not achieved within the typical session duration, the system may propose an extension of the session length so that the subject receives required therapeutic efficacy.

FIG. 4D depicts another example of stimulation burst 432, where interburst intervals e.g., 432a, 432b and 432c may be used to record local neural data including field potentials (LFPs) or event related potentials (ERPs) as a measure of dose response for a session of stimulation. In this example, a set of train sequences is delivered on a relatively slow basis (e.g., every 10 seconds). If, for example, the neural data recorded during the interburst interval 432c indicates that a sufficient dose has been received, therapy might be terminated early. The scheduling of subsequent therapy sessions, as well as their anticipated duration, may be dynamically adjusted based on the dosing outcomes of the current session. For example, if a morning session achieves neural measures indicating a dose response within 50% of the expected duration, the system may either delay a lunchtime session to account for the above-average therapeutic effect or reduce the expected duration of the lunchtime session to 50%. This adjustment may enable the subject to better plan for the timing and delivery of the therapy.

The neural measurements derived from the recorded data during the interburst intervals to observe target engagement may be processed either independently, one at a time, or simultaneously across multiple intervals, depending on the specific therapeutic requirements. To manage complexity, these measurements may be sequenced across different phases within the treatment cycle. For example, at the beginning of a session, the number of electrical pulses may be adjusted for effective initial stimulation. Following this, the sequencing of pulse trains may be fine-tuned to induce specific local field potential (LFP) changes. Finally, the pulse trains may continue until a more significant goal, such as a shift in event-related potentials (ERPs) or a broader change in brain state, is achieved. This phased approach may enable the gradual adjustment of stimulation parameters to improve the therapeutic effect while simplifying the overall process.

In another example, the measurement process may operate in reverse, where a session begins with predetermined train and pulse parameters based on prior configurations. Adjustments or improvements are then initiated only if the targeted changes in event-related potentials (ERPs) are not observed, enabling adaptive adjustments to be made in response to the real-time neural data. In all these examples illustrated in FIG. 4D, if the targeted neural response is not achieved even after extending the treatment parameters to their maximum limits—such as increasing pulse counts, extending pulse train durations, or maximizing session timing and dosing, the subject and/or the attending clinician may be notified. This alert indicates that the therapeutic objectives have not been met, and follow-up actions or adjustments are required.

In certain aspects, once the stimulation parameters 410 are initially set—either through the closed-loop techniques described with reference to FIG. 4, or by the clinician—those parameters may be dynamically adjusted to maintain target engagement. In other aspects, the disclosed techniques may provide a measure of target engagement that enables the clinician to fine-tune the stimulation parameters 410 or stimulation dose to achieve a targeted level of engagement. The techniques for maintaining target engagement are conceptually similar to those used for improving target engagement, with the primary distinction being the shift in objective—from improving engagement to improving its sustained maintenance over time. Maintaining target engagement may be particularly a concern due to the changing tissue environment around the DOT device 108 and burr hole 124 over the course of time following implantation. Initially, the implant site is surrounded by saline, which is rapidly replaced by a protein-rich immune response within hours. This is subsequently replaced by encapsulation tissue, which eventually regrows into the skull over a period of time e.g., several months. Each of these tissue environments has distinct electrical properties, which may affect the efficiency of stimulation. Further, this environment may continue to change as the brain age or other neurodegenerative processes proceed. Therefore, while effective stimulation at the time of dosing may be optimal shortly after implantation, the same stimulation parameters 410 may not produce the targeted effects after months, or even years, due to the evolving tissue characteristics.

The disclosed techniques may address this issue by continuously recording biophysical signals (or biophysical data 206) to monitor target engagement and automatically adjusting stimulation parameters 410 to compensate for changes in the tissue environment for consistent target engagement over time. Furthermore, closed-loop stimulation techniques may be utilized to adapt to changes in the brain itself, such as those arising from synaptic reorganization or neuroplasticity, thus maintaining effective target engagement as the brain undergoes natural changes.

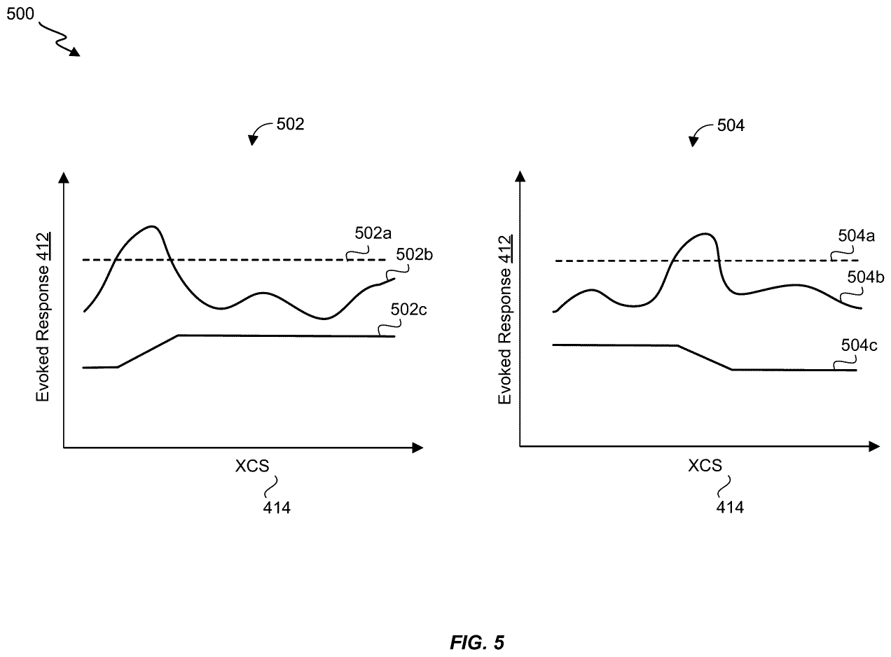

FIG. 5 illustrates exemplary graphs 500 for a closed-loop neuromodulation technique that is configured to maintain target engagement by dynamically adjusting the stimulation parameters 410. The x-axis represents the XCS (or titrated stimulation dose) 414, which may be controlled by a specific stimulation parameter (e.g., amplitude, pulse width, number or pattern of stimulation pulses, frequency and the like). The y-axis represents the evoked (neural) response 412, which is modulated by the titrated stimulation dose 414 and captured from the neural data 402. In this specific example, a single-threshold model is implemented, where the evoked response 412 is continuously monitored to avoid overshooting from a specified threshold 502a and accordingly stimulation parameters are adjusted. In other words, the neural response 502b or the evoked response 412 may be guided by controlling a stimulation parameter 502c (e.g., pulse width, amplitude and the like) that in turn controls the titrated stimulation does 414 in a closed-loop. This single-threshold model may be particularly useful when neural signal 502b correlates with neural engagement or therapeutic outcome.

It may be observed from a graph 502 that initially the neural signal 502b (or the evoked response 412) causes the controlled stimulation parameter 502c to increase and rise above a specified threshold 502a to maintain neural engagement. As the neural response 502b falls below the threshold 502a, the controlled stimulation parameter 502c stabilizes maintaining a fixed level. The stimulation parameter 502c may be continuously varied to maintain the neural response 502b, thereby enabling at least a minimum engagement of the target but also avoiding too much engagement of the target which may represent wasted energy or the risk of a side effect. By using real-time feedback from the recorded neural activity, this closed-loop system maintains the stimulation below the specified threshold 502a, preventing overstimulation. Similarly, for graph 504, the neural response 504b crosses a threshold 504a, causing the stimulation parameter to decrease from a stabilized state. Consequently, as the neural response 504b decreases, the stimulation parameter 504c stops decreasing and maintains a fixed value. This technique may be particularly useful if the neural response 504b correlates with side effects.

In some aspects of the present disclosure, the techniques are disclosed for reducing side effects that may come in the form, but are not limited to, aberrant neural activity, scalp sensations, twitching, heart rate changes, and blood pressure changes. For reducing the side effects, the biological data 214, including the physiological data 210 and biophysical data 206, may be incorporated, for example, EMG electrodes on the scalp placed directly over the XCS stimulator or electrodes place on the scalp facing surface of the DOT may detect muscle twitching and with the right filter setting neural activation indicating scalp sensation. Similarly, ECG or wearables may detect changes in heart rate or heart rate variability, and optical sensors may detect blood pressure or blood flow changes. These streams of data may be integrated by the controller 408, as illustrated in FIG. 4A, to adjust stimulation parameters 410 to reduce side effects while also providing robust therapeutic stimulation.

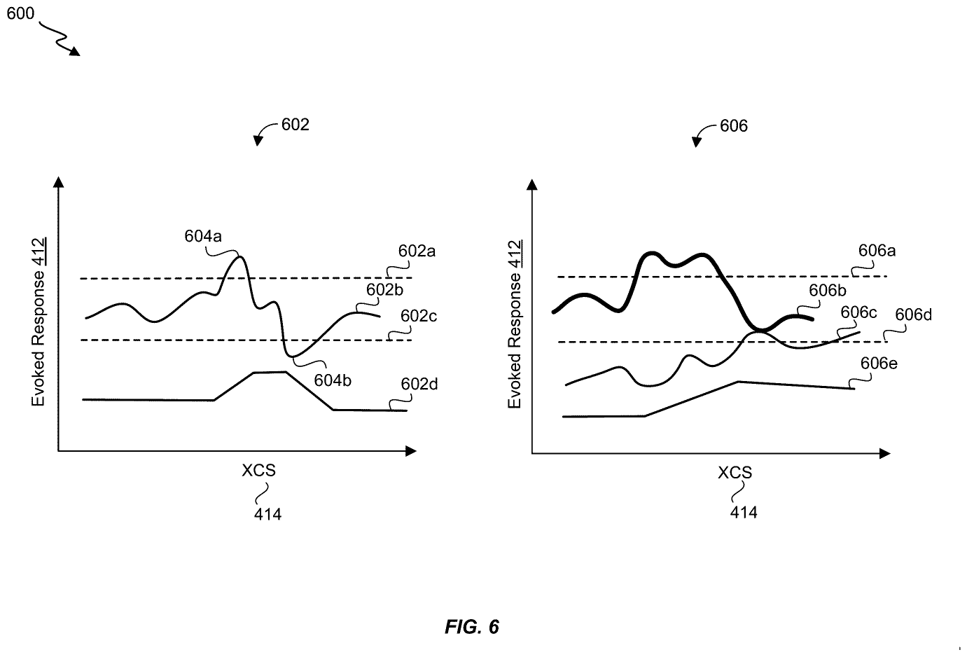

FIG. 6 illustrates exemplary graphs for minimizing the side effects of the disclosed neuromodulation techniques, in accordance with some aspects of the present disclosure. In this specific example, a dual-threshold model is implemented, where the evoked response 412 is continuously monitored to maintain a targeted range and accordingly a stimulation parameter (e.g., 602d or 606e) is adjusted. In a graph 602, two different peaks (e.g., 604a and 604b) may be identified in the neural response 602b occurring at different times and a stimulation parameter 602d may be adjusted such that a ratio of the two peaks is kept within a targeted range. In this example, the targeted range may be defined by a first threshold 602a that defines an upper boundary and a second threshold 602c that defines a lower boundary of the targeted range. The first threshold 602a may correlate with useful target engagement and a second threshold 602c may correlate with a negative side effect such as muscular contraction in the scalp. By continuously adjusting the stimulation parameter 602d such that the first is maximized and the second minimized, the subject may receive a targeted level of stimulation at all times while reducing risk of negative side effects. In other examples, the measure of side effects may not be present in the neural response 602b and may instead be detected by worn sensors providing heart rate, EMG, or other physiological parameters.

For managing conditions such as mania and depression, a dual-threshold model may be leveraged for maintaining a balance between targeted stimulation and reducing side effects of neuromodulation therapy. For example, in mania, neural activity tends to be higher, with heightened brain activity in areas (or cortical regions) responsible for mood regulation and arousal, potentially leading to symptoms e.g., hyperactivity or impulsivity. On the other hand, depression is characterized by lower neural activity, particularly in areas such as the prefrontal cortex, resulting in symptoms e.g., low energy, sadness, and cognitive impairment. The dual-threshold model may address this balance by continuously monitoring the neural response 602b and adjusting stimulation parameters 410 so that brain activity stays within a targeted (or optimal) range. As an additional example, these neural features of mania and depression may be present at one location in the brain, or at two locations monitored by separate DOTs at each target. As such, a neural measure at one location may be combined with a neural measure in a second location to determine stimulation adaptation at a third location, or any such combination of sources of neural signals and targets of neural stimulation across a set of implanted DOT devices.

For the graph 602, the first threshold 602a may correspond to the higher levels of neural activity associated with mania, while the second threshold 602c may align with the reduced neural activity seen in depression. By setting and maintaining these thresholds, the system may adjust stimulation to keep the brain activity within a targeted range—enhancing engagement during depressive states (where activity is too low) while avoiding overstimulation in manic episodes (where neural activity is too high). This dual-threshold approach may enable that the therapy is fine-tuned to avoid side effects, such as excessive neural excitation, while still addressing the underlying disorder.

In another graph 606 of the FIG. 6, a first neural response 606b representing efficacy crosses a first threshold 606a, causing a stimulation parameter 606e to increase. A second neural response 606c representing the side effect crosses a second threshold 606d, causing the stimulation parameter 606e to decrease. The stimulation parameter is controlled by both responses e.g., by a ratio or other relationships. This dual-threshold model may be particularly useful when the therapeutic outcome continually balances efficacy with side effects.

These examples of controlling a stimulation parameter are among those known in the art as being useful for adjusting therapeutic stimulation based on a neural or physiological signal of interest. Beyond simple threshold-based models, other control policies may also be implemented, such as proportional-integral-derivative (PID) controllers. PID controllers work by adjusting stimulation parameters based on three components: the proportional response (which corrects the error based on the current deviation from the target state), the integral response (which accounts for accumulated past errors over time), and the derivative response (which predicts future errors by evaluating the rate of change). When applied to neural data, a PID controller may continuously adjust stimulation based on real-time feedback from neural recordings, fine-tuning the stimulation to reach the targeted brain state while reducing error.

In contrast, neural controllers typically use more complex machine-learning models or neural networks, processing time-series data streams (neural recordings) as inputs. These systems adapt dynamically to changes in neural activity, using the data to generate control signals that adjust stimulation parameters. While both PID controllers and neural controllers aim to optimize therapeutic outcomes, PID controllers use a more structured, rule-based approach, whereas neural controllers rely on adaptive learning techniques to adjust the stimulation based on real-time neural patterns.

In more complex applications, it is possible to envision cases where multiple stimulation parameters are adjusted simultaneously in response to a single neural or biological signal, enabling multi-dimensional control. Alternatively, two separate signals may be used to adjust two distinct stimulation parameters concurrently, enhancing the therapy by targeting different aspects of the neural or biological state. This approach may provide flexibility to fine-tune the therapy more precisely for real-time adaptation to the evolving conditions of the subject and enhancing the therapeutic outcome. Through such advanced control strategies, the stimulation can be tailored to more accurately guide neural or physiological states toward a desired therapeutic goal, accounting for both the immediate responses and longer-term changes in the subject's condition.

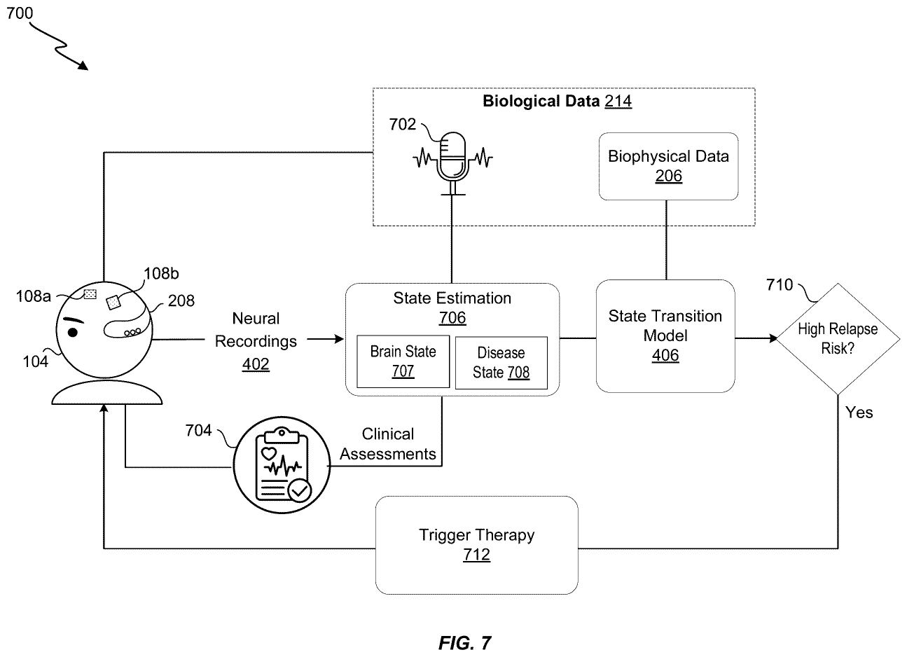

FIG. 7 illustrates an exemplary block diagram 700 for predicting a relapse risk for a subject 216 undergoing a neuromodulation therapy, in accordance with some aspects of the present disclosure. Relapses, in this context, may refer to the recurrence or worsening of symptoms that the therapy was intended to address, such as the return of a neurological or psychological condition after a period of improvement. The relapse risk may refer to a likelihood or a probability that the subject will experience a recurrence or worsening of symptoms or condition after a predefined period of time following the neuromodulation therapy. The relapse risk may be based on the response of the subject to the therapy, as well as other factors such as physiological, psychological, or environmental conditions that may influence the stability of the subject's condition. In certain situations, where relapse risk is identified, the closed-loop XCS therapy may be initiated for the subject to help prevent relapsing.

The process for predicting risks of relapses may include capturing the neural data 402, which may be collected through the implanted devices e.g., DOTs to monitor brain activity of the subject. In addition to DOTs, these neural data 402 may be collected from the biophysical data 206 of the subject, for example, techniques such as electroencephalography (EEG) 206a, functional magnetic resonance imaging (fMRI), and near-infrared spectroscopy (NIRS) may also be employed to monitor brain activity. EEG 206a may provide real-time measurements of electrical brain activity, useful for detecting abnormalities in neural patterns, while fMRI may offer detailed images of brain regions involved in specific cognitive tasks. NIRS is a non-invasive method that measures brain oxygenation levels, which may be indicative of neural activity changes associated with mental states.

Based on the neural data 402, brain state estimation 404 may be performed, similar to the techniques of enhancing target engagement. However, brain state estimation 404 may not be limited to neural data 402 alone. Clinical assessments 704, such as the HAM-D (Hamilton Depression Rating Scale) and MADRS (Montgomery-Åsberg Depression Rating Scale), may be integrated to evaluate the overall condition of the subject. These assessments may help determine a disease state 708, which reflects a detailed evaluation of both the mental and physical symptoms associated with the specific disease being treated with XCS therapy. The disease state estimation 706 may contribute to predicting relapse risk, as it provides a better picture of the subject's health, taking into account not only the mental symptoms but also the physical manifestations that contribute to the overall condition of the subject. These assessments, alongside neural data 402, may help determine the likelihood of symptom recurrence or worsening over a predefined period of time following neuromodulation therapy. These clinical assessments 704 may be used to measure depression severity, providing a deeper picture of the condition of the subject 216, considering both mental health and physical symptoms.

Incorporating speech recordings 702 into this process may add another layer of information that may help in predicting the overall condition (or disease state) of the subject. Audio devices (microphones or speakers) track vocal patterns that may reveal subtle changes in emotional state, cognitive function, or mood. For example, variations in speech rate, tone, and fluency may serve as early indicators of relapse before clinical symptoms become more pronounced. In conjunction with brain state estimations 404 and clinical assessments 704, speech recordings 702 may provide valuable insights into the disease state 708 of the subject. This combination may help monitor the well-being of the subject in real time, potentially predicting relapse risk and enabling timely adjustments to therapy to prevent further deterioration. In some examples, speech recordings 702 may be analyzed by breaking down various components, such as word choice, tone, and cadence, to gain insights into the disease state of the subject. These speech features may be particularly useful because these features may reveal subtle shifts in emotions, cognitive functioning, and overall mental health, which may not always be captured through clinical assessments alone. By continuously monitoring these speech patterns, along with other neural data 402, the system can assess how the subject's disease state is evolving in real-time.

The disease state 708, representing the current overall condition of the subject, may include a severity of symptoms and the likelihood of a relapse. By comparing the real-time brain state estimation 404 with the subject's speech patterns and clinical assessments 704, it may be identified when the disease state 708 shifts to a level that has a high likelihood of relapse. This might be reflected in the subject's worsening symptoms, changes in speech patterns (such as slower speech or less coherent thoughts), or altered brain activity suggesting an impending crisis. When a high risk of relapse is predicted e.g., at 710, XCS (externally powered electrical cortical stimulation) therapy may be triggered e.g., at 712, delivering targeted stimulations to stabilize the brain and prevent further deterioration. These closed-loop neuromodulation techniques may enable timely intervention, adjusting treatment to maintain remission and reduce the chances of a relapse for the subject's condition remains under control over time.

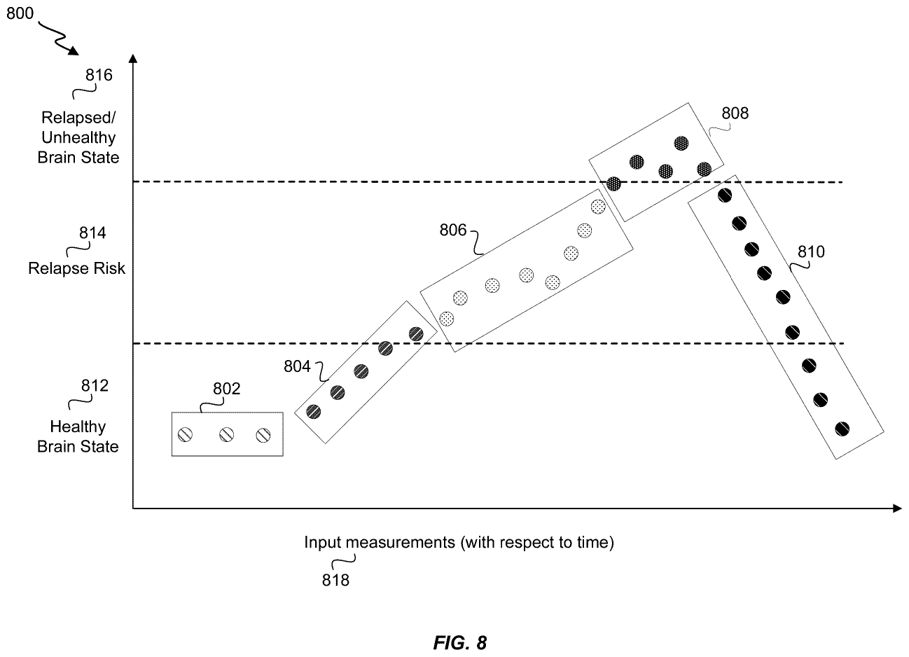

FIG. 8 illustrates an exemplary graph 800 of time-series progression of brain states derived from input measurements 818 including periodically sampled neural data and non-neural data to dynamically adjust therapeutic modes in response to the changes in brain states. For the exemplary curve 800, the x-axis corresponds to the input measurements 818 over a given period of time, while the y-axis represents different brain states, ranging from a healthy brain state 812, to an intermediate relapse risk 814, to relapsed/unhealthy brain state 816. To quantify neural signatures of different brain states, various metrics such as brain wave frequencies (e.g., alpha, beta, and theta), functional connectivity, and event-related potentials (ERPs) are extracted from the neural data such as EEG or fMRI. Predefined thresholds, established from both normative and clinical data, may assist in classifying these states. For example, in a healthy brain state, alpha wave power (8-12 Hz) would be balanced, while beta waves (13-30 Hz) would reflect active cognition and emotional regulation. In contrast, a relapse risk state might show slight disruptions e.g., increased theta waves (4-8 Hz) and reduced prefrontal cortex connectivity, indicating early signs of vulnerability. An unhealthy or relapsed brain state may exhibit more pronounced abnormalities, such as lower alpha power and significant disruption in brain network connectivity, particularly in areas involved in self-regulation and decision-making.

These thresholds may be determined through extensive research and analysis of data from both healthy individuals and those at risk or in relapse, enabling accurate classification. By monitoring changes in these metrics over time, shifts in brain states may be tracked with machine-learning techniques that are often used to automate and refine the process. As neural signatures deviate from the baseline healthy state, these signatures help identify risk levels, enabling early intervention before full relapse occurs.

In the FIG. 8, various data points (e.g., 802, 804, 806, 808 and 810) may depict measured brain states to indicate different phases of therapy intervention or therapy modes. The therapy modes may comprise, e.g., monitoring 802, increasing risk 804, relapse risk 806, relapse 808, and returning to monitoring 810. For each different therapy mode, one or more stimulation parameters corresponding to the stimulation dose (or XCS) may vary. The data points may be derived from the neural and non-neural inputs, which may be processed using analytical methods such as machine-learning classifiers, statistical thresholding, or signal processing techniques. For example, the neural inputs may include neural data 402 e.g., local field potentials (LFPs) recorded from implanted XCS devices, electroencephalography (EEG) signals tracking large-scale brain activity, spiking activity from intracortical electrodes indicating neural firing patterns, and evoked potentials that assess brain network responses to stimuli (e.g., XCS or external stimulus). These neural signals may provide direct insight into functional brain states and their fluctuations.

In addition to neural data 402, external physiological and behavioral measures may contribute to estimation of brain state and an overall disease state. Physiological data 210 e.g., including heart rate variability (HRV) as a marker of autonomic nervous system activity, galvanic skin response (GSR) reflecting stress or arousal, and respiratory patterns that may correlate with certain mental health conditions. Behavioral and cognitive assessments, such as motor activity tracking via accelerometers, speech and language analysis using natural language processing (NLP), and cognitive task performance metrics, may further refine brain state classification. These multimodal data sources may be combined using feature extraction and classification techniques to generate a robust representation of brain state, which may then be plotted on the y-axis.

Based on the measured brain states, the system dynamically shifts between different therapy modes. When the brain state remains within the healthy brain state range 812, the system remains in a monitoring mode 802, where only baseline data collection or minimal maintenance therapy is applied. As the brain state approaches the risk of relapse threshold, the system transitions to increasing risk, where monitoring frequency may increase, and minor therapeutic adjustments may be introduced to stabilize the state. If further deterioration is detected, the system may enter relapse risk state 814, where treatment intensity is adjusted—this may involve changes in stimulation therapy parameters such as dose intensity, duration, or frequency, as well as engagement of caregivers or clinical support. If the brain state crosses into the relapse/unhealthy brain state region 816, the system may activate relapse mode of therapy, delivering full therapeutic intervention with maximum neural monitoring and clinical engagement. Once recovery is achieved, the therapy mode transitions to returning to monitoring mode 810, enabling stability before scaling down interventions. This closed-loop approach may enable real-time therapeutic adaptation, reducing the relapse risk 814 while improving long-term subject outcomes.

The decision to transition between therapy modes may be based on multiple factors. Threshold-based triggers may enable predefined limits—calibrated from population data or individual subject history—to dictate mode changes. Alternatively, machine-learning models may predict relapse risk 814 based on historical patterns, enabling proactive intervention before the state deteriorates further. In some cases, clinical oversight may be required, so that therapy adjustments align with expert judgment. Overall, FIG. 8 demonstrates a data-driven, closed-loop therapeutic approach where brain state estimation (or disease state estimation)—integrating neural, physiological, and behavioral inputs—may guide dynamic treatment adaptations. By continuously monitoring and predicting brain state changes, therapy may be maintained precisely targeted and responsive, improving long-term treatment efficacy while reducing the likelihood of relapse.

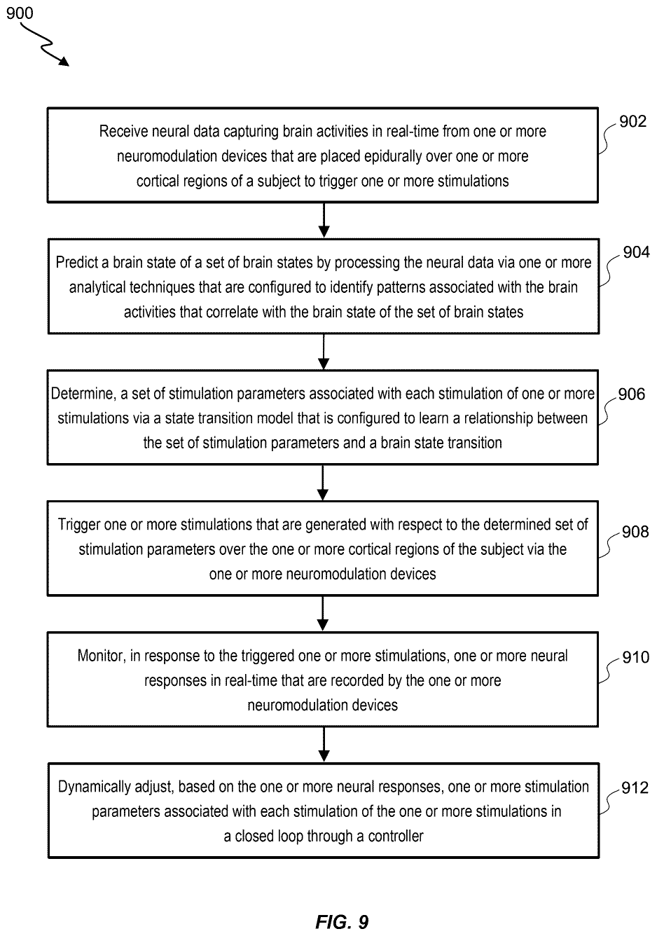

FIG. 9 illustrates an exemplary workflow 900 that relates to dynamic closed-loop neuromodulation techniques for triggering externally powered electrical cortical stimulations (XCS) configured to perform various treatment therapies based on neural data of a subject. The treatment therapies may be targeted to achieve enhanced target engagement, maintaining the target engagement, reducing side effects of the therapy (e.g., twitching), and preventing a relapse. The blocks in the workflow 900 are illustrated in a specific order, while the order can be modified, for example, some blocks may be performed before others, and some blocks may be performed simultaneously. At block 902, the neutral data of the subject may be received in real time from one or more neuromodulation devices (e.g., 108a and 108b). The neuromodulation devices, capturing brain activities, may be placed epidurally over one or more cortical regions to trigger stimulations (interchangeably used herein with XCS). In some aspects, the one or more neuromodulation devices are surgically implanted into the skull 112 of the subject via a bur hole procedure. The neuromodulation devices may be wirelessly powered by one or more magnetic coils 202, configured externally within a headset 208. At block 904, a brain state 707 may be predicted by processing the neural data via one or more analytical techniques that are configured to identify brain activities patterns that correlate with the different brain states e.g., including a healthy state, relapse risk state, an unhealthy state, or other functional modes.

Based on the predicted brain state 707, a set of stimulation parameters 410 may be determined associated with each stimulation of one or more stimulations via a state transition model 406 that may configure to learn a relationship between the set of stimulation parameters 410 and a brain state transition from one brain state 707 to another over time based on the ongoing neural activity (or the neural data), at block 906. This model may learn from past brain states 704 to understand how different stimulation parameters 410 (e.g., intensity, pulse width, frequency) affect brain state transitions and corresponding stimulation outcomes. At block 908, one or more stimulations may be generated with respect to the set of stimulation parameters 410, which may then be triggered over the one or more cortical regions of the subject via the one or more neuromodulation devices (e.g., 108a, . . . , 108e). Each stimulation may be configured to perturb or modulate the brain activities with respect to treatment therapy (or an XCS therapy).

In some aspects of the present disclosure, one or more neural responses in response to the triggered one or more stimulations (or XCS) may be monitored from the neural data, at block 910. These neural responses may be collected in real-time via the one or more implanted neuromodulation devices. At block 912, based on the neural responses, the one or more stimulation parameters 410 may be dynamically adjusted for each stimulation of the one or more stimulations via controller 408 that may be integrated within a headset. These adjustments may be configured to perform the treatment therapy by guiding the brain activities through targeted transitions, using the insights gained from the learned state transition model 406. Additionally, in some aspects, the state transition model 406 may integrate baseline features 308 and evoked features 310 derived from biophysical data 206. This integration may enhance the ability of the state transition model 406 to map neurocardiac coupling and brain state 707 from extracranial recordings, both of which may be incorporated into the state transition model 406 to improve target engagement and the precision of stimulation parameters 410.

Although specific aspects have been described, various modifications, alterations, alternative constructions, and equivalents are possible. Embodiments are not restricted to operation within certain specific data processing environments but are free to operate within a plurality of data processing environments. Additionally, although certain aspects have been described using a particular series of transactions and steps, it should be apparent to those skilled in the art that this is not intended to be limiting. Although some flowcharts describe operations as a sequential process, many of the operations can be performed in parallel or concurrently. In addition, the order of the operations may be rearranged. A process may have additional steps not included in the figure. Various features and aspects of the above-described aspects may be used individually or jointly.

Further, while certain aspects have been described using a particular combination of hardware and software, it should be recognized that other combinations of hardware and software are also possible. Certain aspects may be implemented only in hardware, or only in software, or using combinations thereof. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation, such as by executing computer instructions or code, or processors or cores programmed to execute code or instructions stored on a non-transitory memory medium, or any combination thereof. Processes can communicate using a variety of techniques including but not limited to conventional techniques for inter-process communications, and different pairs of processes may use different techniques, or the same pair of processes may use different techniques at different times.

Specific details are given in this disclosure to provide a thorough understanding of the aspects. However, aspects may be practiced without these specific details. For example, well-known circuits, processes, algorithms, structures, and techniques have been shown without unnecessary detail in order to avoid obscuring the aspects. This description provides example aspects only, and is not intended to limit the scope, applicability, or configuration of other aspects. Rather, the preceding description of the aspects can provide those skilled in the art with an enabling description for implementing various aspects. Various changes may be made in the function and arrangement of elements.

The specification and drawings are, accordingly, to be regarded in an illustrative rather than a restrictive sense. It can, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

What is claimed is:

1. A method comprising:
receiving neural data capturing brain activities in real-time from one or more neuromodulation devices that are placed epidurally over one or more cortical regions of a subject to trigger one or more stimulations, wherein a neuromodulation device of the one or more neuromodulation devices is surgically implanted into a skull of the subject via a bur hole procedure;
predicting a brain state of a set of brain states by processing the neural data via one or more analytical techniques that are configured to identify patterns associated with the brain activities that correlate with the brain state of the set of brain states;
determining, based on the predicted brain state, a set of stimulation parameters associated with each stimulation of one or more stimulations via a state transition model that is configured to learn a relationship between the set of stimulation parameters and a brain state transition;
triggering, via the one or more neuromodulation devices, the one or more stimulations over the one or more cortical regions of the subject, wherein the one or more stimulations are generated with respect to the determined set of stimulation parameters, and wherein the one or more stimulations are configured to perturb the brain activities with respect to a treatment therapy;
monitoring one or more neural responses in real-time recorded by the one or more neuromodulation devices in response to the triggered one or more stimulations; and
dynamically adjusting, based on the one or more neural responses, one or more stimulation parameters associated with each stimulation of the one or more stimulations in a closed loop via a controller, wherein the adjustment is configured with respect to the treatment therapy by guiding the brain activities through targeted brain state transitions that are learned from the state transition model, and wherein the treatment therapy includes enhancing target engagement, maintaining target engagement, reducing side effect, and preventing relapse.

2. The method of claim 1, further comprising:
determining a candidacy of the subject for applying the one or more stimulations with respect to the treatment therapy, wherein the candidacy is determined by:
collecting a first neural data from the subject capturing brain activities in real-time;
collecting a second neural data from the subject in response to an external stimulus that is configured to collect the second neural data non-invasively by placing one or more stimulation electrodes over the one or more cortical regions of the subject, wherein the external stimulus includes a transcranial magnetic stimulation (TMS) or a transcranial electrical stimulation (TES);
generating one or more baseline features from the first neural data and one or more evoked features from the second neural data; and
predicting, based on the one or more baseline features and the one or more evoked features, a subject candidacy that corresponds to a likelihood of the subject to benefit from a neuromodulation therapy based on a classification model that is trained on historical data from past subjects that were exposed to external stimuli with known outcomes.

3. The method of claim 1, wherein the neural data includes one or more of: local field potentials (LFPs), stimuli-evoked potentials (SEP), and event-related potentials (ERPs).

4. The method of claim 1, further comprising:
inputting one or more baseline features and one or more evoked features into the state transition model, wherein:
the one or more baseline features are derived from biophysical data in absence of a stimulus and the one or more evoked features are derived from the neural data in presence of the stimulus, and
the state transition model includes a Hidden Markov Model (HMM).

5. The method of claim 1, further comprising:
collecting biological data of the subject that comprises one or more of: neural data, cardiovascular data, respiratory data, movement and activity data, environmental data, demographic data, anthropometric data, comorbidities data, medical imaging data, lifestyle indicators data, feedback or a combination thereof; and
dynamically adjusting, based on the biological data, the one or more stimulation parameters associated with each stimulation of the set of stimulations in the closed loop via the controller, wherein the adjustment is configured to reduce side effects of the treatment therapy while delivering the one or more stimulations.

6. The method of claim 1, wherein the set of brain states includes a healthy brain state, a relapse risk brain state and an unhealthy/relapse brain state.

7. The method of claim 1, further comprising:
recording the one or more neural responses during an interburst interval associated with a triggered stimulation of the one or more triggered stimulations that comprises one or more bursts of pulses with predefined amplitudes and interburst intervals;
determining, based on the recorded one or more neural responses, that a targeted neural response is achieved with respect to the treatment therapy; and
dynamically terminating, in response to the determination, the triggered one or more stimulations.

8. The method of claim 1, wherein the controller is configured externally within a headset that includes one or more magnetic coils configured to wirelessly power the one or more neuromodulation devices.

9. The method of claim 1, wherein the one or more neuromodulation devices are configured to exchange the neural data and the neural response in real-time using radio frequency protocol with each other and the controller to dynamically adjust the one or more stimulations.

10. A system comprising:
one or more processors;
one or more non-transitory computer-readable media storing instructions, which, when executed by the system, cause the system to perform a set of actions including:
receiving neural data capturing brain activities in real-time from one or more neuromodulation devices that are placed epidurally over one or more cortical regions of a subject to trigger one or more stimulations, wherein a neuromodulation device of the one or more neuromodulation devices is surgically implanted into a skull of the subject via a bur hole procedure;
predicting a brain state of a set of brain states by processing the neural data via one or more analytical techniques that are configured to identify patterns associated with the brain activities that correlate with the brain state of the set of brain states;
determining, based on the predicted brain state, a set of stimulation parameters associated with each stimulation of one or more stimulations via a state transition model that is configured to learn a relationship between the set of stimulation parameters and a brain state transition;
triggering, via the one or more neuromodulation devices, the one or more stimulations over the one or more cortical regions of the subject, wherein the one or more stimulations are generated with respect to the determined set of stimulation parameters, and wherein the one or more stimulations are configured to perturb the brain activities with respect to a treatment therapy;
monitoring one or more neural responses in real-time recorded by the one or more neuromodulation devices in response to the triggered one or more stimulations; and
dynamically adjusting, based on the one or more neural responses, one or more stimulation parameters associated with each stimulation of the one or more stimulations in a closed loop via a controller, wherein the adjustment is configured with respect to the treatment therapy by guiding the brain activities through targeted brain state transitions that are learned from the state transition model, and wherein the treatment therapy includes enhancing target engagement, maintaining target engagement, reducing side effect, and preventing relapse.

11. The system of claim 10, wherein the set of actions further includes:
determining a candidacy of the subject for applying the one or more stimulations with respect to the treatment therapy, wherein the candidacy is determined by:
collecting a first neural data from the subject capturing brain activities in real-time;
collecting a second neural data from the subject in response to an external stimulus that is configured to collect the second neural data non-invasively by placing one or more stimulation electrodes over the one or more cortical regions of the subject, wherein the external stimulus includes a transcranial magnetic stimulation (TMS) or a transcranial electrical stimulation (TES);
generating one or more baseline features from the first neural data and one or more evoked features from the second neural data; and
predicting, based on the one or more baseline features and the one or more evoked features, a subject candidacy that corresponds to a likelihood of the subject to benefit from a neuromodulation therapy based on a classification model that is trained on historical data from past subjects that were exposed to external stimuli with known outcomes.

12. The system of claim 10, wherein the neural data includes one or more of: local field potentials (LFPs), stimuli-evoked potentials (SEP), and event-related potentials (ERPs).

13. The system of claim 10, wherein the set of actions further includes:
inputting one or more baseline features and one or more evoked features into the state transition model, wherein:
the one or more baseline features are derived from biophysical data in absence of a stimulus and the one or more evoked features are derived from the neural data in presence of the stimulus, and
the state transition model includes a Hidden Markov Model (HMM).

14. The system of claim 10, wherein the set of brain states includes a healthy brain state, a relapse risk brain state and an unhealthy/relapse brain state.

15. The system of claim 10, wherein the one or more neuromodulation devices are configured to exchange the neural data and the neural response in real-time using radio frequency protocol with each other and the controller to dynamically adjust the one or more stimulations.

16. A computer-program product tangibly embodied in a non-transitory machine-readable storage medium, including instructions configured to cause one or more data processors to perform a set pf actions including:
receiving neural data capturing brain activities in real-time from one or more neuromodulation devices that are placed epidurally over one or more cortical regions of a subject to trigger one or more stimulations, wherein a neuromodulation device of the one or more neuromodulation devices is surgically implanted into a skull of the subject via a bur hole procedure;
predicting a brain state of a set of brain states by processing the neural data via one or more analytical techniques that are configured to identify patterns associated with the brain activities that correlate with the brain state of the set of brain states;
determining, based on the predicted brain state, a set of stimulation parameters associated with each stimulation of one or more stimulations via a state transition model that is configured to learn a relationship between the set of stimulation parameters and a brain state transition;
triggering, via the one or more neuromodulation devices, the one or more stimulations over the one or more cortical regions of the subject, wherein the one or more stimulations are generated with respect to the determined set of stimulation parameters, and wherein the one or more stimulations are configured to perturb the brain activities with respect to a treatment therapy;
monitoring one or more neural responses in real-time recorded by the one or more neuromodulation devices in response to the triggered one or more stimulations; and
dynamically adjusting, based on the one or more neural responses, one or more stimulation parameters associated with each stimulation of the one or more stimulations in a closed loop via a controller, wherein the adjustment is configured with respect to the treatment therapy by guiding the brain activities through targeted brain state transitions that are learned from the state transition model, and wherein the treatment therapy includes enhancing target engagement, maintaining target engagement, reducing side effect, and preventing relapse.

17. The computer-program product of claim 16, wherein the set of actions further includes:
determining a candidacy of the subject for applying the one or more stimulations with respect to the treatment therapy, wherein the candidacy is determined by:
collecting a first neural data from the subject capturing brain activities in real-time;
collecting a second neural data from the subject in response to an external stimulus that is configured to collect the second neural data non-invasively by placing one or more stimulation electrodes over the one or more cortical regions of the subject, wherein the external stimulus includes a transcranial magnetic stimulation (TMS) or a transcranial electrical stimulation (TES);
generating one or more baseline features from the first neural data and one or more evoked features from the second neural data; and
predicting, based on the one or more baseline features and the one or more evoked features, a subject candidacy that corresponds to a likelihood of the subject to benefit from a neuromodulation therapy based on a classification model that is trained on historical data from past subjects that were exposed to external stimuli with known outcomes.

18. The computer-program product of claim 16, wherein the neural data includes one or more of: local field potentials (LFPs), stimuli-evoked potentials (SEP), and event-related potentials (ERPs).

19. The computer-program product of claim 16, wherein the set of actions further
includes:
inputting one or more baseline features and one or more evoked features into the state transition model, wherein:
the one or more baseline features are derived from biophysical data in absence of a stimulus and the one or more evoked features are derived from the neural data in presence of the stimulus, and
the state transition model includes a Hidden Markov Model (HMM).

20. The computer-program product of claim 16, wherein the set of brain states includes a healthy brain state, a relapse risk brain state and an unhealthy/relapse brain state.
